(19) United States
(12) Patent Application Publication
(10) Pub. No.: US 2026/0182991 A1
(43) Pub. Date: Jul. 2, 2026

(54) DEVICES TO REDUCE FLOW INTO A VASCULAR DEFECT

(71) Applicants: Nhan Thai, Anaheim, CA (US); Ulysses Grant, Tustin, CA (US); Parker Milhous, Santa Ana, CA (US); Jose Guerra Garcia, Corona, CA (US); Brent Fritz, Costa Mesa, CA (US); Matthew Fitz, San Juan Capistrano, CA (US)

(72) Inventors: Nhan Thai, Anaheim, CA (US); Ulysses Grant, Tustin, CA (US); Parker Milhous, Santa Ana, CA (US); Jose Guerra Garcia, Corona, CA (US); Brent Fritz, Costa Mesa, CA (US); Matthew Fitz, San Juan Capistrano, CA (US)

(73) Assignee: Nuvascular Inc, Irvine, CA (US)

(21) Appl. No.: 19/006,182

(22) Filed: Dec. 30, 2024

Related U.S. Application Data
(60) Provisional application No. 63/623,471, filed on Jan. 22, 2024.
(60) Provisional application No. 63/635,842, filed on Apr. 18, 2024.

(51) Int. Cl. A61B 17/12; A61B 17/00; A61B 90/00

(52) U.S. Cl. CPC A61B 17/12113; A61B 17/12172; A61B 90/39; A61B 2017/00836; A61B 2017/00862; A61B 2017/00867; A61B 2017/00946; A61B 2017/1205; A61B 2090/3966

(57) ABSTRACT

Devices and methods for treatment of a patient's vasculature including occluding aneurysms and blood vessels are described. The device includes a self-expanding resilient permeable shell having a radially constrained state and an expanded state with an axially shortened configuration. The permeable shell may be a single layer of braided elongate filaments having first and second ends that are secured at the proximal end of the permeable shell. The devices may also include permeable shells made of woven braided mesh having a variable mesh density, i.e., the average size of pores in one region are a different than the average size of pores in another region. Devices and methods of using the device to treat a cerebral aneurysm are also described.

### RELATED APPLICATIONS

This application claims priority from U.S. Provisional Application Ser. No. 63/623,471 filed Jan. 22, 2024 and U.S. Provisional Application Ser. No. 63/635,842 filed Apr. 18, 2024. All of the above applications are herein incorporated by reference in their entirety for all purposes.

### TECHNICAL FIELD

This application relates to implantable medical devices to occlude blood vessels and/or aneurysms within a patient's body. This may be medically necessary to, for example, stop pathological bleeding into an end organ such as a spleen or ovary. Some embodiments of the instant invention relate to the treatment of vascular defects such as aneurysms, including cerebral aneurysms. Aneurysms may have weak, thin walls that can rupture, resulting in disability or death. Aneurysms in the brain may be treated by excluding the aneurysm from the parent blood vessel by, for example, surgical clipping, packing the aneurysm with coils, or by using stents to divert or reduce flow into the aneurysm. Another method of treating aneurysms is to place a braided basket or woven device into the aneurysm to reduce blood flow and promote endothelization at the neck of the aneurysm.

### BACKGROUND

Some methods of obstructing blood flow into a blood vessel or aneurysm involve placing braided, implantable medical devices into the blood vessel or the aneurysm. Additional background information may be found in U.S. Pat. No. 10,478,194; U.S. Pat. No. 10,130,372; U.S. Pat. No. 10,265,075; U.S. Pat. No. 10,939,914; U.S. Pat. No. 11,058,431; US2023/0200817 A9, and WO2023081340A1; all incorporated herein by reference.

In some aspects, prior art may describe implantable devices having proximal and distal sections with additional material or higher pore density at the proximal section to reduce or occlude blood flow and less dense areas at the distal part of the device. Some embodiments of the instant invention describe implantable devices having a dense distal section near an open distal end, giving a counterintuitive and unexpected result of increasing the radial force exerted by the distal section of the device on the wall of the aneurysm near an equatorial area or within a blood vessel, thus improving the stability of the device to resist movement caused by the pulsatile blood flow and/or by manipulation during detachment of the implantable device.

Some aspects of prior art may describe implantable devices for treating aneurysms or occluding blood vessels in which the devices are formed by folding one or more layers of braided material or placing multiple layers of braid on top of each other or placing multiple braided implants inside of each other. Multiple layers of braid may increase the density of braid in the aneurysm neck and may make the device more occlusive. Although any of the implant designs described herein could be made from multiple layers or by folding over a single-layer braided tube, and such designs are within the scope of this patent, and any of the embodiments that follow could be understood and created by one skilled in the art by substituting the term “single-layer” with “multilayer”; the multiple layer or folded devices may require larger delivery catheters compared to a single-layer design or a design with fewer layers. Thus, there exists a long-felt but unsolved need for an implantable occlusive device that provides the additional density of braided material achievable with multiple layers of braided material to improve occlusion at the aneurysm neck while being capable of delivery through a small catheter. Some embodiments of the instant invention describe implantable devices configured for delivery through a catheter or microcatheter as a single layer device, the device capable of forming multiple layers when delivered to a treatment site.

### SUMMARY

Embodiments of a device to slow or occlude blood flow into a vascular defect such as an aneurysm or a blood vessel feeding, for example, a bleeding organ or tumor, are described. In some embodiments, the device may be configured as an intrasaccular device to treat aneurysms occurring, for example, in the brain. In some embodiments, the device may be configured to be placed in an approximately cylindrical blood vessel such as an artery or vein to slow blood flow within the vessel.

Some embodiments of an implantable device for treatment of a patient's vasculature may include a self-expanding resilient permeable shell having a radially constrained state configured for delivery within a catheter lumen, an expanded state, and a plurality of elongate filaments which are braided or woven together. Some embodiments may comprise a woven braided mesh implant wherein the implant has an open distal end, a distal region or section adjacent to the distal end, a proximal region, one or more intermediate region(s) between the distal and proximal regions, and a proximal end adjacent to the proximal region. The distal end may comprise a series of loops or arcs around the open circumference formed from the elongate filaments. Alternatively, the open distal end may be formed by cutting the elongate filaments. Alternatively, the distal end may be formed by a combination of looped and cut filaments. The distal region adjacent to the distal end may comprise a single layer or multilayer woven braided mesh defining a circumferential diameter. The filaments of the woven braided mesh define diamond-or rhombus-shaped pores wherein the rhombus-shape defines a circumferential axis and a longitudinal axis and wherein each pore has a length along the circumferential axis, a length along the longitudinal axis and a pore area that is approximately the product of the circumferential length and the longitudinal length divided by two. The longitudinal length of at least one pore in the distal region may be less than the circumferential length of the same pore. An intermediate region is adjacent to the distal region also comprises a woven braided mesh with diamond-or rhombus-shaped pores having circumferential and longitudinal lengths and a pore area when the implant is in an expanded configuration. When the implant is in the expanded configuration, the area of at least one pore in the intermediate region is less than the area of at least one pore in the adjacent distal region. Additionally, in the expanded configuration, the intermediate region defines a circumferential diameter that is less than the circumferential diameter defined by the distal region. The implant may comprise additional intermediate regions in the expanded configuration, the additional intermediate region(s) having a braided mesh construction and defining diamond-or rhombus-shaped pores with longitudinal and circumferential lengths with pore areas approximately half the product of the longitudinal and circumferential lengths, wherein the pore area of at least one of the pores may be larger than the pore area of at least one of the pores in the distal region.

In some embodiments, the intermediate region(s) between the first intermediate region and the proximal region define circumferential diameter(s), the circumferential diameter(s) of the intermediate region(s) may be less than the circumferential diameter of the distal region. Additionally, the longitudinal length of a pore in an intermediate region(s) may be equal to or less than the circumferential length of the same pore. A proximal region formed from a woven braided mesh is proximally adjacent to an intermediate region. In the expanded configuration, the proximal region defines a circumferential diameter which may be larger than the circumferential diameter of the adjacent intermediate region. In the expanded configuration, the circumferential diameter of the proximal region may be larger than at least one of the intermediate region(s). When the implant is in the expanded configuration, the proximal region comprises diamond-or rhombus-shaped pores with longitudinal and circumferential lengths, the pores having a pore area approximately equal to the product of the longitudinal and circumferential lengths divided by two. The area of at least one pore in the proximal region may be approximately equal to or smaller than the area of at least one pore in the distal region. The longitudinal length of at least one pore in the proximal region may be less than the circumferential length of the same pore. At the proximal end of the braided mesh implant, the filaments may be gathered and welded, glued, crimped, soldered, or otherwise jointed together either within an outer joining element of material such as plastic, metal, and/or radiopaque metal or without an outer joining element. The proximal end of the device may additionally be configured to be detachably coupled to a delivery system capable of moving the implant from a proximal end of a catheter to a treatment location.

In some embodiments, devices for treating cerebral aneurysms are described. These embodiments may include an implant comprising a woven braided mesh that is substantially open on a distal end and substantially closed on a proximal end and having a series of regions between the distal end and the proximal end as described above. The implant is configured to be placed within a cerebral aneurysm having a dome and a wall, the aneurysm dome having a first diameter and a neck having a second diameter. The distal end of the implant may be non-obstructive to blood flow and may be configured for placement near the first diameter of the aneurysm dome. The implant comprises a closed proximal region that may obstruct blood flow proximally adjacent to at least one intermediate region, wherein the device is configured so that the proximal region expands from a radially constrained state within a catheter to an expanded state near the neck of the aneurysm wherein the closed proximal region may obstruct blood flow at the neck of the aneurysm. At least one intermediate region may be configured to conform to the aneurysm wall. The proximal region defines a circumferential diameter. The diameter of the proximal region in an expanded state may be approximately the same diameter as the aneurysm neck diameter, or proximal region diameter in an unrestrained expanded configuration may be approximately up to 4 mm larger than the aneurysm neck diameter, or the proximal region diameter in an unrestrained expanded configuration may be approximately 0% to 20% larger than the aneurysm neck diameter.

In some embodiments, devices for occluding blood vessels are described. These devices may include an implant comprising a woven braided mesh having a substantially open distal end, a substantially closed proximal end, and a series of regions between the distal end and the proximal end as described above. In some embodiments, the implant is configured to be placed within a blood vessel having a diameter. The implant comprises a substantially closed proximal region that may obstruct blood flow. The device may be configured so that the proximal region expands from a radially constrained state within a catheter to an expanded state within the blood vessel. The diameter of the proximal region in an expanded state may be approximately the same diameter as the blood vessel diameter, or proximal region diameter in an unrestrained expanded configuration may be approximately up to 2 mm larger than the vessel diameter, or the proximal region diameter in an unrestrained expanded configuration may be approximately 1% to 20% larger than the vessel diameter.

Some embodiments of implantable devices configured to treat cerebral aneurysms or blood vessels, may include a self-expanding resilient permeable shell having a radially constrained elongated state configured for delivery within a catheter lumen, an expanded state with a longitudinally shortened configuration relative to the radially constrained state, and a plurality of elongate filaments which are braided or woven together. The device may include a woven braided mesh implant wherein the implant has a substantially open distal end section, a distal region adjacent to the distal end section, a proximal region, one or more intermediate region(s) between the distal and proximal regions, and a substantially closed proximal end adjacent to the proximal region. The distal end section comprises a series of loops or arcs formed from the elongate filaments to form an open circumference. The distal region adjacent to the distal end comprises a woven braided mesh defining a circumferential diameter when the implant is in an expanded configuration. The filaments of the woven braided mesh define diamond-or rhombus-shaped pores wherein the pore shape defines a circumferential axis and a longitudinal axis and wherein each pore has a length along the circumferential axis, a length along the longitudinal axis and a pore area. The longitudinal length of at least one pore in the distal region may be less than the circumferential length of the same pore. An intermediate region is adjacent to the distal region also comprises a woven braided mesh with diamond-or rhombus-shaped pores having circumferential and longitudinal lengths and a pore area when the implant is in an expanded configuration. When the implant is in the expanded configuration, the area of at least one pore in the intermediate region is less than the area of at least one pore in the adjacent distal region. Additionally, in the expanded configuration, the intermediate region defines a circumferential diameter that is less than the circumferential diameter defined by the distal region. The implant may comprise additional intermediate regions in the expanded configuration, the additional intermediate region(s) having a braided mesh construction and defining diamond-or rhombus-shaped pores with longitudinal and circumferential lengths with pore areas, wherein the pore area of at least one of the pores may be larger than the pore area of at least one of the pores in the distal region. Additionally, the intermediate region(s) between the first intermediate region and the proximal region define circumferential diameter(s), the circumferential diameter(s) of the intermediate region(s) may be less than the circumferential diameter of the distal region. Additionally, the longitudinal length of a pore in an intermediate region(s) may be equal to or greater than the circumferential length of the same pore. A proximal region formed from a woven braided mesh is proximally adjacent to an intermediate region. In the expanded configuration, the proximal region defines a circumferential diameter which may be larger than the circumferential diameter of the adjacent intermediate region. In the expanded configuration, the circumferential diameter of the proximal region may be larger than at least one of the intermediate region(s). In the expanded configuration, the circumferential diameter of the proximal region may be approximately 30% to 80% of the circumferential diameter of the distal region. When the implant is in the expanded configuration, the proximal region comprises diamond-or rhombus-shaped pores with longitudinal and circumferential lengths, the pores having a pore area. The area of at least one pore in the proximal region may be approximately equal to or smaller than the area of at least one pore in the distal region. The longitudinal length of at least one pore in the proximal region may be less than the circumferential length of the same pore. At the proximal end of the braided mesh implant, the filaments may be gathered and welded, glued, crimped, soldered, or otherwise jointed together either within an outer joining element of material such as plastic, metal, and/or radiopaque metal or without an outer joining element. The proximal end of the device may additionally be configured to be detachably coupled to a delivery device capable of moving the implant from a proximal end of a catheter to a treatment location.

In some embodiments of devices for occluding aneurysms, wherein the aneurysm to be treated has a dome having an approximate diameter and a neck having an approximate diameter, some embodiments may comprise an implantable mesh formed from one or more elongate filaments. The mesh may be formed into an implant from a single layer or multiple layers of braided or woven filaments. The implant may comprise a distal end of loops or arcs formed by looping one or more filaments over a post or beam, forming an open-ended structure having a distal diameter. In an expanded configuration, the distal diameter of the open-ended structure may be approximately equal to 100% to 200% of the diameter of the aneurysm's dome diameter. The implant may define a longitudinal axis through approximately the center of the distal diameter. The mesh implant may define a closed proximal end in which one or more elongate filaments are gathered by, for example, welding, soldering, gluing, or encasing within an appropriately cylindrical joining element. The longitudinal axis approximately defined by the distal diameter may pass through the proximal end of the implant and define a longitudinal length from the distal end of the implant to the proximal end of the implant. Between the distal end and the proximal end, the mesh implant in an expanded configuration may define one or more cross-sectional diameter(s) perpendicular to the longitudinal axis. Each cross-sectional diameter may be smaller than the distally adjacent cross-sectional diameter for a first portion of the device until a minimum cross-sectional diameter is defined by the mesh implant. The minimum cross-sectional diameter may be 10%-80% of the distal diameter. The location of the minimum cross-sectional diameter may be 10%-90% of the longitudinal length measured from the distal end to the proximal end. The cross-sectional diameter defined by the mesh implant proximally adjacent to the minimum cross-sectional diameter may be larger than the minimum cross sectional diameter. Additional proximally adjacent cross-sectional diameter(s) may be larger than the previous cross-sectional diameter(s). A proximal cross-sectional diameter near the proximal end may be larger than the minimum cross-sectional diameter. A cross-sectional diameter defined by the mesh near the proximal end may be larger than the minimum cross-sectional diameter. A cross-sectional diameter defined by the mesh near the proximal end may be 20% to 80% smaller than the distal diameter. A cross-sectional diameter defined by the mesh near the proximal end of the implant in an expanded configuration may be 0% to 30% larger than the approximate diameter of the neck of the aneurysm.

Some embodiments of devices for slowing or occluding blood flow into an aneurysm or blood vessel comprise any of the above described device wherein, in an expanded state, elongate filament(s) of a woven braided mesh implant define(s) at least one pore at or near a minimum cross-sectional diameter of the implant, the minimum cross-section diameter section's pore(s) having a longitudinal length, a circumferential length, and an area. In addition, the elongate filament(s), in an expanded state, define at least one pore at or near a proximal cross-section diameter wherein the pore(s) at or near the proximal diameter has a longitudinal length, a circumferential length, and an area, wherein the area of the pore(s) at or near the cross-sectional minimum diameter of the implant are larger than the area of the pore(s) at or near the proximal diameter of the implant.

Some embodiments describe devices for slowing or occluding blood flow into a vascular defect, some embodiments may comprise an implant formed of one or more layers of woven braided mesh wherein the woven braided mesh is formed from one or more elongated filament(s). In an expanded state, implant comprises a first substantially open end, a second substantially closed end, and a central axis. The first end may comprise an open-ended series of loops or arcs formed from bending the filament(s). The second end is formed by gathering the filament(s) and joining the filament(s) by laser welding, soldering, gluing, and/or mechanical crimping. The woven braided mesh may define a first frustoconical shape having a first diameter in a plane perpendicular to the central axis of the implant. Between the first end and the second end, the woven braided mesh may define a second diameter in plane perpendicular to the central axis wherein the second diameter may be smaller than the first diameter. The second diameter of the woven braided mesh may further define a second frustoconical shape wherein the first frustoconical shape is arranged facing the second frustoconical shape to approximate an asymmetric hourglass shape wherein the first end may be substantially open and the second end may be substantially closed. Between the second diameter and the second end, the woven braided mesh may define a third diameter in a plane perpendicular to the central axis wherein the third diameter may be larger than the second diameter. Between the second diameter and the second end, the woven braided mesh may define a third diameter in a plane perpendicular to the central axis wherein the third diameter may be larger than the second diameter and wherein the third diameter may be smaller than the first diameter.

Some embodiments describe devices for slowing or occluding blood flow into a vascular defect, some embodiments may comprise an implant formed of one or more layers of woven braided mesh wherein the woven braided mesh may be formed from one or more elongated filament(s). The woven braided mesh implant may define a self-expanding resilient permeable shell having a central axis, an outer surface, an inner surface, a distal end, and a proximal end. Additionally, the self-expanding resilient permeable shell may have a radially constrained elongated state configured for delivery within a catheter lumen, an expanded state with a longitudinally shortened configuration relative to the radially constrained state, and one or more elongate filament(s) forming the mesh.

### BRIEF DESCRIPTION OF THE DRAWINGS

Some representative embodiments are illustrated in the drawings and description in which similar elements are assigned the same reference numerals. However, while some embodiments are illustrated in the drawings, there is no intention to limit the instant invention to the specific embodiments or embodiments disclosed. Rather, the present invention is intended to cover all modifications, alternative constructions, and equivalents falling withing the spirit and scope of the invention. As such, the drawings are intended to be illustrative and not restrictive. Preferred embodiments of the present invention are described as non-limiting examples only, with respect to the accompanying drawings in which:

FIG. 1 shows an embodiment of a device for slowing or occluding blood flow into an aneurysm or a blood vessel.

FIG. 1A is a cross-section of FIG. 1 with the radii and central axis of an embodiment shown.

FIG. 2 shows a pore configuration of a braided shell.

FIG. 3 shows an embodiment of a device for occluding an aneurysm or blood vessel.

FIG. 4 shows an embodiment of a device for occluding an aneurysm or blood vessel.

FIG. 4A is a cross-section of FIG. 4 with the diameters of an embodiment shown.

FIG. 5 shows an embodiment of a device for occluding an aneurysm or blood vessel.

FIG. 6 shows an embodiment of a device for occluding an aneurysm or blood, the device vessel capable of changing conformations.

FIG. 6A illustrates a first and second conformation of the embodiment shown in FIG. 6.

FIG. 7 shows an embodiment configured for delivery through a catheter.

FIG. 8 shows delivery of an implant into an aneurysm.

FIG. 9 shows an embodiment of a device and method for closing the flange during deployment.

FIG. 10 shows an embodiment of a device for occluding an aneurysm or blood vessel, the device capable of changing conformations.

FIG. 11 illustrates a conformation change in dashed lines with some layers of braided material not shown for clarity.

FIG. 12 illustrates a multi-tiered embodiment of a device for occluding an aneurysm or blood vessel having multiple pore densities and diameters.

FIG. 13 shows an embodiment of a single-layer, braided device with an open distal end and a closed proximal end, the device having multiple sections with some sections shaped like saucers, cylinders, and frustoconical shapes, each shape having a pore density and a diameter.

FIG. 13A shows a top view of the device of FIG. 13 highlighting the overlapping layers of material wherein a multilayer device may be formed from a single-layer braided device.

FIG. 14 shows a device deployed within a simulated aneurysm having a diameter larger than the height of the aneurysm wherein a top layer is flipped over an intermediate layer.

FIG. 15 illustrates a single layer braided device having an open distal end, a disk or saucer shaped distal section, a cylindrical intermediate section, and a closed proximal section having a higher pore density than the intermediate section.

FIG. 16 shows a bi-stable or multi-stable embodiment in the non-flipped configuration.

FIG. 17 shows a bi-stable or multi-stable embodiment in the flipped configuration.

FIG. 18 illustrates an embodiment comprising a single layer of braided material with an open distal end comprising loops or apices folded or bent toward a central axis, a distal section, a cylindrical intermediate section, and closed proximal section.

FIG. 19 shows an embodiment comprising a single layer of braided material with an open distal end comprising loops or apices folded or bent toward a central axis, a disk shaped distal tier, a intermediate section in the approximate shape of two inverted trapezoids, and a disk-shaped proximal tier with a pore density greater than the pore density of the intermediate section.

FIG. 20 shows an embodiment comprising a single layer of braided material with an open distal end comprising loops or apices and multiple sections that may be defined by bends or curves, wherein each section may define a diameter and/or a length, the sections comprising pores that may have one or multiple sizes, wherein the braided material may be joined at one end by cylindrical member, and wherein the overall length of the device may be defined by the distance between the distal end and a proximal edge of the cylindrical member.

FIG. 21 shows an embodiment comprising a single layer of braided material with an open distal end comprising loops or apices and multiple sections that may be defined by bends or curves, wherein each section may define a diameter and/or a length, the sections comprising pores that may have one or multiple sizes, wherein the braided material may be joined at one end by cylindrical member, wherein multiple radii of curvature may form a path from a proximal section to the cylindrical member.

FIG. 22 shows an embodiment deployed within an aneurysm and an axial force vector of the blood flow (F1) and examples of radial vector forces (F2 and F3).

### DETAILED DESCRIPTION OF EMBODIMENTS

Unless otherwise defined, technical terms used herein have the same meaning as commonly understood by one of ordinary skill in the art to which this technology belongs. The terms “region” and “section” may be used interchangeably to refer to a portion of the device. The terms “woven” and “braided” are used interchangeably to mean any form of interlacing of filaments to form a mesh structure. The term “filament”, “filaments”, and “elongate filament(s)” are used interchangeably to mean a wire, fiber, or thread of any shape including, but not limited to, round, square, oval, rectangular, or ovoid made from a variety of metallic or plastic materials including, but not limited to, alloys or Drawn Filled Tubes (DFT) of nickel titanium (Nitinol), platinum, iridium, tungsten, tantalum, gold, chromium cobalt (Elgiloy or MP35), stainless steel, polyolefin, PET, Dacron, hydrogel, or any combination thereof. The terms “expanded”, “expanded state”, “free air”, “unconstrained”, and “unconstrained state” are used interchangeably to refer to an implantable device having self-expanding properties in which the device deployed without being constrained within a treatment site such as an aneurysm, blood vessel, or other vascular malformation. The term “pore” refers to an open (metal free) area formed during a weaving or braiding process by the crossing(s) of filament(s). By convention, pore(s) are defined by a perimeter measured from the inside edge of the wire or filament. Pore(s) could also be measured from the outside edge of the wire and the diameter of the filament subtracted, or the pore(s) could be measured from a centerline passing through the center of the wire or filament. The term “pore density” means the approximate number of pores per square millimeter or square inch of surface area of a braid.

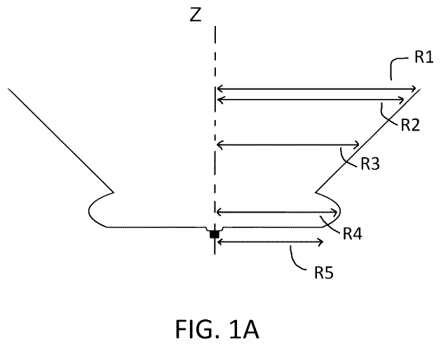

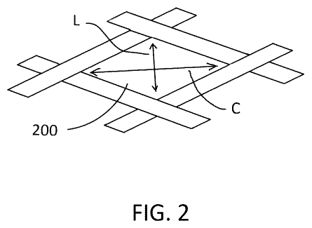

An implantable device 10 shown in FIG. 1 in an expanded state comprises a self-expanding single layer, or multi-layer, permeable shell 40 braided from filaments 70. A variety of methods may be employed to form the shell 40, including machine braiding techniques described in U.S. Pat. No. 10,939,914 and in Braiding Technology for Textiles ISBN 978-0-08-101329-8, both incorporated herein by reference. The device 10 defines a central axis Z, a distal end 20, at least one distal region 35, at least one intermediate region 45, at least one proximal region 50, and a proximal end 60. The distal end 20 may comprise a series of loops or curved portions of filament 30 which may be formed by looping a series of filaments around a pin, post, groove, or similar feature of a braiding mandrel and braiding the filaments around the braiding mandrel. In some embodiments, some, or all, of the loops 30 may be cut with, for example, scissors or a laser. As shown in FIG. 1A, the distance from the central axis Z to an outer edge of the distal end 20 defines a radius R1 the device 10. In some embodiments, R1 is approximately 10% to 90% larger than the radius of a treatment site such as the radius of a blood vessel or aneurysm sac. The first distal region 35 lies adjacent to the distal end 20, the distance between the central axis Z and the outer edge of first distal region 35 defines a radius R2. In some embodiments, R2 is less than R1. In other embodiments, there may be a bulge within the first distal region 35 in which R1 is less than R2. FIG. 2 shows a representative pore 200 of the permeable shell 40 formed from filaments 70. The pore of has a longitudinal length L and a circumferential length C that approximately perpendicular to L. The length of L and C vary along the length of the device 10, with the length L of any particular region denoted as L1, L2, . . . Ln and the length of C denoted as C1, C2, . . . , Cn.

The first distal region 35 comprises a braid having at least one pore, the pore defining a longitudinal axis L2 parallel to the central axis Z and a circumferential axis C2 perpendicular to the longitudinal axis. In some embodiments, device 10 may comprise additional distal regions adjacent to the first distal region 35, each distal region having a radius and comprising a braid having at least one pore. The pore(s) in the first distal region 35 may be formed by filaments 70 such that the length(s) of the pore(s) in the circumferential axis C2 is (are) longer than the length(s) of the pore(s) in the longitudinal axis L2. In some embodiments, C2 is 20% to 150% longer than L2. Adjacent to the first distal region 35 or any additional distal region(s), the device 10 comprises at least one intermediate region 45 between the distal region 35 and the proximal region 50. The intermediate region 45 may be defined by multiple planes perpendicular to the central axis Z each having a radius generically shown in FIG. 1A as R3, but which may encompass multiple radii, wherein each plane had an inscribed cross-section of the intermediate region 45. In some embodiments, the inscribed cross-section of the intermediate region 45 defines a circular cross section having a circumference. In some embodiments, each circumference of each plane in the intermediate region 45 may be the same size or smaller than the distally adjacent circumference defined in the distally adjacent plane. The intermediate region comprises a woven braided shell 40 having at least one pore, the pore defining a longitudinal axis L3 parallel to the central axis Z and a circumferential axis C3 perpendicular to the longitudinal axis. In some embodiments, the pores in the distal region 35 define an area approximately equivalent to half the product of L2 and C2 and the pores in the intermediate region 45 define an area approximately equivalent to half the product of L3 and C3. In some embodiments, the area of at least one pore in the distal region 35 is less than the area of at least one pore in the intermediate region 45. In some embodiments, the area of a pore in the distal region 35 is 20% to 90% of the area of a pore in intermediate region 45. In some embodiments, within the intermediate region 45, the pores in one plane perpendicular to the central axis Z may have a shorter circumferential length compared pores in a proximally adjacent parallel plane. The proximal region 50 is proximally adjacent to the intermediate region 45. In some embodiments, the proximal region 50 comprises a braid having at least one pore, the pore defining a longitudinal axis L4 parallel to the central axis Z and a circumferential axis C4 perpendicular to the longitudinal axis. In some embodiments, the pores in the proximal region 50 define an area approximately equivalent to half the product of L4 and C4. In some embodiments, the area of at least one pore in the proximal region 50 is less than the area of at least one pore in the intermediate region 45. In some embodiments, the area of a pore in the proximal region 50 is 20% to 90% of the area of a pore in intermediate region 45. In some embodiments, the distance between the central axis Z and the distal edge 80 of the proximal region 50 defines a radius R4. In some embodiments, the distance between the central axis Z and an edge 90 of the proximal region 50 proximally adjacent to the distal edge 80 of the proximal region 50 defines a radius R4. In some embodiments, the distance between the central axis Z and a proximal edge 100 of the proximal region 50 defines a radius R5. In some embodiments, R3 is less than R4. In some embodiments, R5 is less than R4. In some embodiments, R3 is less than R4. In some embodiments, intermediate region 45 may define multiple radii, some of the radii may be larger than R4 and some radii may be smaller than R4. In some embodiments, R5 is less than R4. In some embodiments, the proximal region 50 comprises a braided shell forming a bulge near the proximal end 60 of the device 10. In some embodiments, the proximal region 50 comprises a braided shell forming a bulge near the proximal end 60 of the device 10, wherein said bulge is configured to be placed at the neck of an aneurysm. In some embodiments the unconstrained radius of the bulge is 0.5 mm to 4 mm larger than the radius of neck of the aneurysm. In some embodiments, the bulge is 10% to 50% larger than the radius of the neck of the aneurysm.

In some embodiments, the device 10 is configured for deployment within an aneurysm to slow or block flow near the neck of the aneurysm. Depending on the size of the aneurysm, the device is formed from 40-400 nickel titanium alloy or chromium cobalt alloy wires ranging from 0.0004 to 0.003 inches diameter and having a radiopaque core material such as platinum or gold comprising 10-40% of the wires'cross sectional area. Wires of this type may be purchased from Fort Wayne Metals under the trade name Drawn Filled Tube (DFT) wire. The wires are looped around pins or slots at the end of a braiding mandrel so that the wires form looped ends 30 at the distal end 20 of the device 10. The braiding mandrel may have a similar shape to the final configuration of the implant or may be a tubular shape and the braided shell 40 may be formed around a shaping mandrel in subsequent operations to form the device 10. In some embodiments, a braided shell 40 is formed on a shaped mandrel having a first radius R1 approximately 1.5 mm to 12 mm, sized to treat aneurysms with diameters approximately 2 mm to 20 mm.

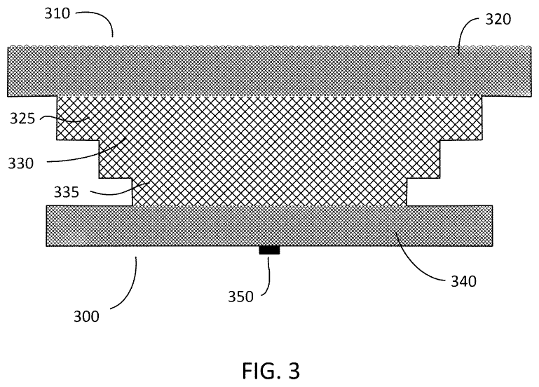

FIG. 3 shows a device having an inverted layer cake, ziggurat, or energy dome-shaped braided shell with at least one larger-diameter layer near a proximal end, the shell forming an implantable device 300 with multiple layers or levels approximately perpendicular to a vertical central axis. The device 300 comprises a distal end 310, a first distal section 320 comprising a braided shell having a first diameter, the braided shell having a first pore density. The implantable device 300 further comprises multiple intermediate layers 325, 330, and 335. In some embodiments, there may be a single intermediate layer. In some embodiments there may be 2 to 10 intermediate layers. In some embodiments there may be more than 10 intermediate layers. The intermediate layers each define a diameter. In some embodiments, the diameter of an intermediate layer may be the same or smaller than the first diameter defined by the distal section 320. In some embodiments, the diameters of the intermediate layers become progressively smaller between the distal section 320 and a proximal braided shell 340. The intermediate layers 325, 330, and 335 each comprise a braided shell, each section of braided shell having a pore density. In some embodiments, the first pore density of the distal section 320 is equal to or greater than the pore densities of the intermediate layers 325, 330, and 335. The proximal section 340 comprises a braided shell having a diameter. In some embodiments, the diameter of the proximal shell is larger than one or more of the diameters of at least one of the intermediate layers. In some embodiments, the diameter of the proximal section 340 is approximately 0.5 mm to 4 mm larger than the diameter of the treatment site. In some embodiments, the device 300 is configured to be deployed in an aneurysm having a neck wherein the diameter of the proximal section is 0.5 mm to 4 mm larger than the neck of the aneurysm. In some embodiments, the device 300 is 10% to 50% larger than the neck of the aneurysm. In some embodiments, the device 300 is configured to be deployed in a blood vessel having a vessel diameter wherein the diameter of proximal section 340 is 0.5 mm to 4 mm larger than the vessel diameter. In some embodiments, the proximal section 340 is 10% to 50% larger than the vessel diameter. The proximal section 340 comprises a braided shell having a pore density wherein the pore density of the proximal section 340 is equal to or greater than the pore density at least one of the intermediate sections 325, 330, or 335. In some embodiments, the filaments that form the braided shell forming the device 300 may be joined at a proximal end 350 by, for example, laser welding or adhesive bonding the filaments within an approximately cylindrical marker band.

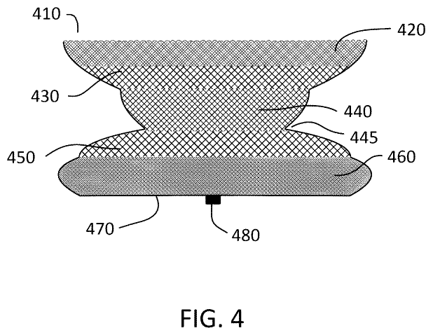

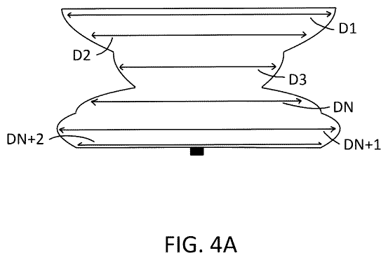

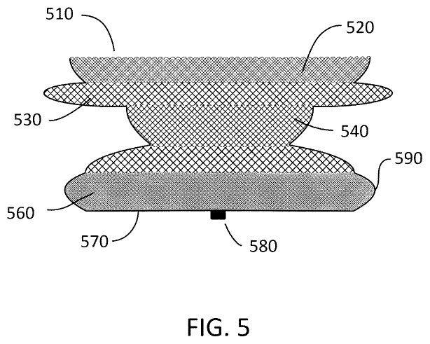

FIG. 4 shows an implantable device comprising multiple frustoconical-shaped horizontal layers or levels pf braided filaments perpendicular to a central vertical axis. The implantable device shown in FIG. 4 comprises a distal end 410 and a proximal end 450. In some embodiments the distal end 410 may comprise loops or arcs formed by wrapping individual filaments around multiple pins at one end of a braiding mandrel. In some embodiments, the distal end 410 may be formed by folding or inverting a tubular braid along a plane approximately perpendicular to the central vertical axis to form a device having two layers of braided material. In alternative embodiments, the distal end 410 may be formed by looping multiple filaments around a single pin affixed to the end of the braiding mandrel, forming a tubular or frustoconical braid, and subsequently cutting the braid along a plane approximately perpendicular to the central vertical axis. A first distal section 420 is proximally adjacent to the distal end 410. The distal section 420 comprises a braided shell having a diameter D1 as shown in FIG. 4A, a pore size, and a pore density. Some embodiments of the implantable device of FIG. 4 and FIG. 4A may further comprise multiple intermediate layers 430, 440, 450. In some embodiments, there may be a single intermediate layer. In some embodiments, the number of layers N may be 2 to 10 intermediate layers. In some embodiments, the number of intermediate layers N may be more than 10. The intermediate levels may each define a diameter D2 . . . DN. In some embodiments, D2 may be smaller than D1. In some embodiments, for example FIG. 5, D2 may be larger than D1, forming an enlarged section proximally adjacent to the distal section. In some embodiments, D3 may be less than D2. In some embodiments, DN may be larger than D3. In some embodiments, the implantable braided device may have multiple horizontal layers with decreasing diameters from the distal region 420 moving toward an intermediate inflection point 445. For illustrative purposes, the intermediate inflection point 445 is shown in FIG. 4 between intermediate layers 440 and 450, although the inflection point 445 could occur at any point between the distal end 410 and the proximal end of the device 450. Proximally adjacent to the intermediate inflection point 445, there may be multiple horizontal layers increasing in diameter up to a proximal region 460. In some embodiments, the proximal region 460 may be formed from a braided shell having a local maximum diameter DN+1. In some embodiments, diameter DN+2 may increase, decrease, or remain approximately the same up to a proximal base 470 of the braided implantable shell. In some embodiments, the base 470 may be formed by gathering the ends of some or all of the filaments forming the braided into a cylindrical band near the proximal end 480. In some embodiments, the base 470 may be formed by gathering the ends of some or all of the filaments forming the braided and joining them together by laser welding or adhesive bonding. In some embodiments, the proximal base 470 may form an approximately flat section with a substantially closed center portion. In some embodiments, the proximal base 470 may form an approximately convex or concave shape. In some embodiments, the proximal end 480 may be adapted to be connected to a detachment system for delivering the implant through a catheter to a treatment site, positioning and repositioning the implant as necessary, and implanting the device at the treatment site.

In some embodiments, the distal section 420 may comprise a braided section. The distal braided section may be formed by braiding one or more filaments around a braiding mandrel to form a tubular braid. During the braiding process, a weighted headpiece may be placed over the filaments, the headpiece urging the filaments against the mandrel as the tubular braid forms. In some embodiments, a first headpiece weight may be used when the distal section 420 is braided to form pores having a pore density P42. The intermediate layers 430, 440, 450 may comprise braided sections having a pore densities P43, P44, and P45 respectively. In some embodiments, proximal section 460 may comprise a braided section or sections having one or more pore densities P46, P46.1, P46.2 . . . P46.N. In some embodiments, during the braiding process, the headpiece weight used when braiding intermediate section 430 may be greater than the first headpiece weight used to form the braid in the distal section 420 in order to form a braided section in 430 wherein the pore density P43 is less than the pore density P42 of the distal section 420. In some embodiments, during the braiding process, the headpiece weight used when braiding intermediate section 440 may be greater than the first headpiece weight used to form the braid in the distal section 420 in order to form a braided section in 440 wherein the pore density P44 is less than the pore density P42 of the distal section 420. In some embodiments, during the braiding process, the headpiece weight used when braiding intermediate section 450 may be greater than the first headpiece weight used to form the braid in the distal section 420 in order to form a braided section in 450 wherein the pore density P45 is less than the pore density P42 of the distal section 420. In some embodiments, during the braiding process, the headpiece weight used when braiding proximal section 460 may be approximately equivalent to the first headpiece weight used to form the braid in the distal section 420 in order to form a braided section in 460 wherein the pore density P46.1 . . . P46.N of the proximal section 460 is approximately the same as the pore density P42 of the distal section 420. In some embodiments, during the braiding process, the headpiece weight used when braiding proximal section 460 may be greater than the first headpiece weight used to form the braid in the distal section 420 in order to form a braided section in 460 wherein the pore density P46.1 . . . P46N of the proximal section 460 is less than the pore density P42 of the distal section 420. In some embodiments, during the braiding process, the headpiece weight used when braiding proximal section 460 may be less than the first headpiece weight used to form the braid in the distal section 420 in order to form a braided section in 460 wherein the pore density P46.1 . . . P46N of the proximal section 460 is greater than the pore density P42 of the distal section 420.

In some embodiments, the diameter of the distal section 420 of the braided shell is larger than one or more of the diameters of at least one of the intermediate layers. In some embodiments, the diameter of the distal section 420 is approximately 0.5 mm to 4 mm larger than the diameter of the treatment site. In some embodiments, the device is configured to be deployed in an aneurysm having a neck wherein the diameter of the proximal section 460 is 0.5 mm to 4 mm larger than the neck of the aneurysm. In some embodiments, the proximal section 460 of the device is 10% to 50% larger than the neck of the aneurysm. In some embodiments, the implantable shell is configured to be deployed in a blood vessel having a vessel diameter wherein the diameter of proximal section 460 is 0.5 mm to 4 mm larger than the vessel diameter. In some embodiments, the proximal section 460 is 10% to 50% larger than the vessel diameter.

FIG. 5 shows an unconstrained implantable device comprising a braided structure formed from one or more layers of braided filaments having a substantially open distal end 510 and a substantially closed proximal end 580. In some embodiments, the device comprises a distal section 520, an intermediate section 530 proximally adjacent to the distal section 520, and a proximal section 560. Some embodiments may comprise one or more intermediate sections generically shown for illustrative purposes as 540. The distal section 520 may define a first diameter and the proximally adjacent intermediate section 530 may define a second diameter. In some embodiments, the first diameter of the distal section 520 may be smaller than the second diameter of the proximally adjacent intermediate section 530. In some embodiments, the braided structure of the distal section 520 may comprise at least one pore wherein the pore(s) has (have) a first circumferential length and a first longitudinal length. In some embodiments, the braided structure of the intermediate section 530 may comprise at least one pore wherein the pore(s) has (have) a second circumferential length and a second longitudinal length. In some embodiments, the first circumferential length of at least one pore within the distal section 520 is less than the second circumferential length of at least one pore within the proximally adjacent intermediate section 530. In some embodiments, the first longitudinal length of at least one pore within the distal section 520 is longer than second longitudinal length of at least one pore within the proximally adjacent intermediate section 530. In some embodiments, the first longitudinal length of at least one pore within the distal section 520 is less than the second longitudinal length of at least one pore within the proximally intermediate section 530. In some embodiments, the braided structure of the distal section 520 comprises multiple pores defining a pore density. In some embodiments, the braided structure of the intermediate section 530 comprises multiple pores defining a pore density. In some embodiments, the pore density of the distal section 520 may be greater than the pore density of the intermediate section 530. In some embodiments, the pore density of the distal section 520 may be less than the pore density of the intermediate section 530. In some embodiments, the pore density of the distal section 520 may be approximately equivalent to the pore density of the intermediate section 530.

In some embodiments, the implantable device may comprise a proximal section 560 that may be proximally adjacent to an intermediate section. The proximal section 560 may comprise a substantially closed end 580 and a proximal surface 570. In some embodiments, the proximal section 560 and proximal surface 570 may be configured to reduce flow at the neck of an aneurysm or reduce flow through a blood vessel. In some embodiments, the proximal section 560 and proximal surface 570 may be formed by braiding filament(s) comprising one or more ends around a substantially cylindrical braiding mandrel and wrapping the resulting tubular structure around a secondary forming mandrel, wherein the secondary forming mandrel comprises a disc shaped section with a curved edge, forming the braided outer edge 590 of the proximal section 560, and the proximal surface 570. After wrapping the tubular braided structure around the secondary forming mandrel, the end(s) of the filament(s) may be gathered into a proximal end 580 and joined by, for example, laser welding, crimping within a metallic cylinder, or adhesive bonding. The proximal section 560 of the implantable device thus formed may comprise a braided, disc-shaped shell having an inner surface, and outer surface, and a proximal surface 570. In some embodiments, the proximal surface 570 may be substantially flat, convex, concave, or may have corrugated or wave shaped appearance.

In some embodiments, the proximal surface 570 may comprise a series of approximately concentric rings of pores formed by the braided filament(s). The pores of a concentric ring may have a circumferential length generically shown in FIG. 2 as C that approximately follows the circumference of the ring and a length approximately perpendicular to C, generically shown in FIG. 2 as L. In some embodiments, the length of C increases as the diameters of the concentric rings increase from a first diameter near the center of the device approximately adjacent to the proximal end 580 to a second diameter near the outer edge 590 of the proximal section 560. In some embodiments, the length L of the pore may increase as circumferential length C of the pore increases. In some embodiments, the length L of the pore may decrease as circumferential length C of the pore increases. In some embodiments, the length L of the pore may remain approximately unchanged as circumferential length C of the pore increases.

In some embodiments, the outer edge 590 of the proximal section 560 may define a diameter wherein the diameter of 590 may be approximately 0.5 mm to 4 mm larger than the neck of the aneurysm or diameter of the blood vessel intended to be treated. In some embodiments, the outer edge 590 of the proximal section 560 may define a diameter wherein the diameter of 590 may be approximately 5% to 75% larger than the neck of the aneurysm or the diameter of the blood vessel intended to be treated.

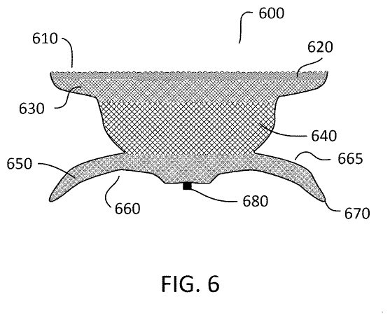

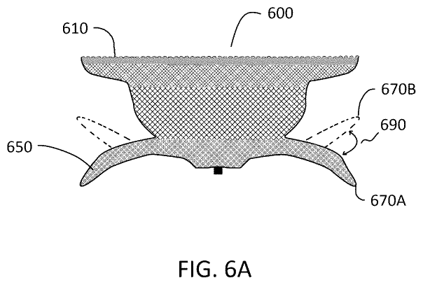

In some embodiments exemplified in FIG. 6, an implantable braided shell device 600, shown in its unconstrained state, may comprise a distal end 610 and a gathering zone 680 where one or more filaments used to construct the braided shell are bonded. In some embodiments, the device 600 may comprise a distal braided section 620. The distal braided section 620 may be formed from braided filaments having a diameter and a pore density, wherein the pore density may be higher (i.e., denser) in the distal section 620 than a first adjacent intermediate braided section 630. The device 600 may comprise a second intermediate braided section 640 proximally adjacent to 630 and having a diameter wherein the diameter of the second intermediate braided section 640 is smaller than the diameter of the distal braided section 620. In some embodiments, the first intermediate section 630 has a diameter that is larger than the proximally adjacent second intermediate section 640. The implantable device 600 may comprise a braided proximal section 650 having a proximal surface 660. The proximal section 650 may be formed into a flange, rim, overhang, projection, extension, lip, or similar protuberance forming a partial or complete circumference (herein referred to as “flange”). In some embodiments, the flange comprises one or more layers of braided filaments and follows an approximately curvilinear path from an intermediate section 630 or 640 to a proximal edge 670. In some embodiments, the flange forms a proximal surface 660. The proximal surface 660 may have an approximately flat, arced, wave-like, or corrugated shape, or a combination of such shapes. In some embodiments, the filaments forming the braided proximal section 650 may be joined in the gathering zone 680 by restraining at least some of the filaments within a hollow cylindrical tube and laser welding or adhesive bonding the filaments together. In some embodiments, in an unconstrained state, the flanged proximal section 650 may curve or slope proximally away from the distal end 610. In some embodiments, in the unconstrained state, the proximal edge 670 may lie in a first plane and the gathering zone 680 may lie in a second plane wherein the first plane may be proximal to the second plane.

In some embodiments, the implantable device 600 may comprise a distal edge 610, a flanged proximal section 650, and a proximal edge 670A as shown in FIG. 6A. The flange may be formed from braided filaments such as nickel-titanium alloy (Nitinol) or Nitinol DFT filaments comprising an outer layer if Nitinol and in inner radiopaque core made from, for example, platinum or gold. As shown in FIG. 6A, the flange may have bistable configurations in which, in a first configuration, the proximal edge 670A is oriented away from the distal edge 610 in a first state and may flip upward as shown in the dotted line in FIG. 6A, into an orientation wherein the proximal edge 670B may be moved to a second configuration at an angle 690 from the first configuration. In some embodiments, the angle between the first flange configuration and the second flange configuration is between 1 degree and 100 degrees. In some embodiments, the angle between the first flange configuration and the second flange configuration is between 15 degrees and 30 degrees. In some embodiments, the angle between the first flange configuration and the second flange configuration is between 30 degrees and 60 degrees. In some embodiments, the angle between the first flange configuration and the second flange configuration is between 45 degrees and 100 degrees.

In some embodiments, the device 600 may comprise a braided shell having an outer surface and an inner surface wherein the proximal edge 670A defines a first plane in a first configuration wherein the first plane lies a first distance from the distal edge 610. The device 600 may comprise a braided shell having an outer surface and an inner surface wherein the proximal edge 670A is configured to change orientation so that the proximal edge may move in a distal direction and may define a second plane in a second configuration, wherein the second plane lies a second distance from the distal edge 610 and wherein the second distance is less than the first distance.

In some embodiments, the device 600 comprises a single layer self-expanding permeable shell having a first unconstrained state and a second radially constrained state configured for delivery within a catheter. In the first unconstrained state, the device comprises braided filaments to form the shell. The shell may comprise a distal end 610 and a proximal gathering section 680 wherein the proximal gathering section may be configured to be detachably connected to a delivery system. The device 600 may comprise a proximal section 650 having a first upper layer of material 665 and a second lower layer of material 660. In the first unconstrained state the proximal section may comprise a protuberance formed from the first single layer of upper material and the second single layer of lower material and may comprise a gap between the first and second layers wherein the gap has a separation distance. In the second radially constrained state, the gap between the layers of braided material comprising the proximal section 650 may increase, the device 600 may axially lengthen, and the separation distance between the first and the second layers forming the protuberance may increase compared to the separation distance between the first and second layers forming the protuberance in the unconstrained state.

In some embodiments, the implantable device comprises a distal end wherein the distal end may comprise a series of castellated loops to form an open circumference. In some embodiments the castellated loops may be formed from one or more filaments braided into a woven shell structure. In some embodiments, the loops at the distal end may be formed by first forming a tubular braid and folding the braid. In some embodiments, the loops at the distal end may be formed by looping at least one elongate filament around a pin or cutout in a braiding mandrel and braiding the looped filament(s) into a braided shell structure. In some embodiments, the device may comprise a distal braided section proximally adjacent to the distal end and an intermediate braided section proximally adjacent to the to the distal braided section wherein the distal braided section may have a pore density that is greater than the pore density of the intermediate section. In some embodiments, the pore density of the distal braided section may be 110% to 400% greater than the pore density of the intermediate braided section. In some embodiments, the pore density of the distal braided section may be 150% to 250% greater than the pore density of the intermediate braided section. In some embodiments, the device may comprise a distal braided section proximally adjacent to the distal end and an intermediate braided section proximally adjacent to the to the distal braided section wherein the distal braided section may have a metal coverage area that is more than the metal coverage area of the intermediate braided section. In some embodiments, the metal coverage area of the distal braided section may by 15%-50% In some embodiments, the metal coverage area of the intermediate braided section may be 5%-30%. In some embodiments, the implantable device may comprise a proximal braided section proximally adjacent to an intermediate braided section wherein the proximal braided section may have a pore density that is greater than the pore density of the intermediate section. In some embodiments, the pore density of the proximal braided section may be 110% to 400% greater than the pore density of the intermediate braided section. In some embodiments, the pore density of the proximal braided section may be 150% to 250% greater than the pore density of the intermediate braided section. In some embodiments, the device may comprise a proximal braided section proximally adjacent to an intermediate braided section wherein the proximal braided section may have a metal coverage area that is more than the metal coverage area of the intermediate braided section. In some embodiments, the metal coverage area of the distal braided section may by 15%-50% In some embodiments, the metal coverage area of the intermediate braided section may be 5%-30%. In some embodiments, the proximal braided section may comprise a protuberance or flange.

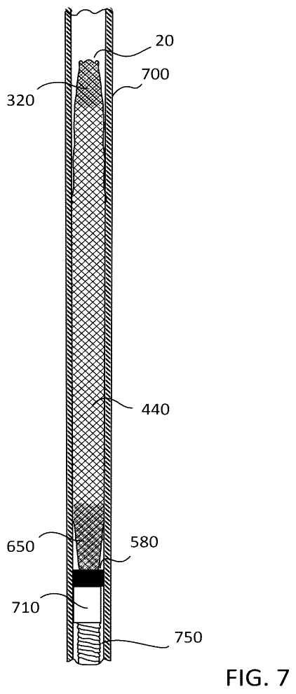

Any of the device embodiments described herein may be radially constrained and configured for deployment through a catheter to an appropriate treatment site as shown in FIG. 7. An implantable device configured from a braided shell comprising, for example, a distal end 20, a distal section 320 having a first pore density, at least one intermediate section 400 having a second pore density, a proximal section 650 having a third pore density, and a gathering section 580 is detachably connected to a detachment mechanism 710 and delivery pusher 750. The implantable device is radially collapsed to constrain it within a delivery catheter 700. Depending on the size of the implant and the target treatment site, the delivery catheter may have an inner diameter from, for example, 0.010 inches to 0.120 inches. In some embodiments configured for treatment of cerebral aneurysms, standard commercially available microcatheters in the 0.015 inch to 0.038 inch range may be used. In applications such as occlusion of peripheral veins or arteries, commercially available catheters in the range of 0.015 inch to 0.088 inches may be used. The delivery catheter or microcatheter may be maneuvered to a treatment site from a femoral or radial artery access point using a guidewire.

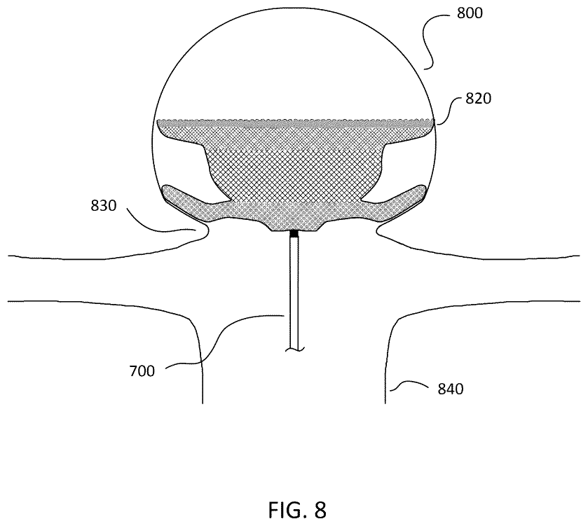

FIG. 8 shows the delivery catheter 700 in position within an artery 840 at a treatment site 800, in this example an aneurysm having a mid-point or equatorial region 820 and a neck region 830. An implant, for example, the implantable device 600, is detachably coupled to a delivery mechanism 710 and configured for deployment within the catheter 700. The device 600 is pushed through the catheter 700 in a radially collapsed state to the treatment site 800 using the delivery pusher 750. For clarity, the aneurysm 800 is shown for illustration purposes to be about the same size as the implant 600. In some embodiments, the equatorial diameter 820 or aneurysm 800, or the diameter of a treatment site such as a blood vessel, may be 5%-75% smaller than the diameter of the distal end 610 of the device 600. In some embodiments, the neck region 830 of the aneurysm 800 would be 1-4 mm smaller or 5%-30% smaller than the outer diameter of the proximal flange 670. In some embodiments, the deployed shape of the implantable device 600 is capable of conforming to the vessel or aneurysm walls, including but not limited to, the aneurysm neck. The device 600 is then deployed within the aneurysm 800 by, for example, pushing the implant out of the delivery catheter 700 with the delivery pusher 750, retracting the delivery catheter 700, or a push/pull combination of moving the delivery catheter and the delivery pusher 750. Once deployed, the implantable device will expand from the radially collapsed state to a radially expanded state which is generally smaller than the free-air or unconstrained state. In some embodiments, the implantable device is configured to conform to the aneurysm wall and/or neck in an expanded state. In some embodiments, the dial end 610 of the implant 600 may contact the wall of the aneurysm 800 approximately near the center or equator of the aneurysm 820. The terms “approximately near” and “equator” and “equatorial” herein should be construed broadly since the equator diameter and location of an aneurysm can vary widely depending on the size and shape of the aneurysm. In some cases, aneurysms are not spherical and may not have a defined center or equator. Generally, however, the terms “equator” and “equatorial” are understood to be near the center as best as can be defined and are distinct from the dome of the aneurysm. In some embodiments the implantable device comprises a resilient braided shell capable of conforming to non-spherical shapes. The size and shape of the implant 600 relative to the size and shape of the aneurysm 800 may cause the implant to deploy at the equator 820, above the equator, or below the equator. As the implant 600 deploys within the aneurysm 800, the distal section 620 may contact the wall of the aneurysm. In some embodiments, the intermediate section(s) 630, 640 may contact the wall of the aneurysm as the implant continues to be deployed from the delivery catheter 700. In some embodiments, the proximal edge 670A of the implant 600 may contact an area approximately near the neck 830 of the aneurysm 800. In some embodiments, as proximal edge 670A encounters the wall or neck of the aneurysm, the proximal edge 670A may move toward the distal end 610 of the implant 600 or away from the delivery catheter 700 toward the dome of the aneurysm 800. In some embodiments, the proximal edge 670 of an implantable device 600 may move or partially move from a first configuration 670A to a second configuration 670B during deployment from a compressed state within a delivery catheter to an expanded state within a treatment site. In some embodiments, the expanded state within the treatment size may be radially compressed relative to unconstrained state. In some embodiments, the proximal section 650 of the implant 600 comprises a proximal edge 670 to form a protuberance or flange. In some embodiments, once deployed into a treatment site, the proximal edge 670 may remain substantially in the orientation of the unconstrained state 670A. In some embodiments, once deployed into a treatment site, the proximal edge 670A may move or “flip” to a new orientation 670B. In some embodiments, once deployed into a treatment site, the proximal edge 670A may move or flip to a new orientation that is an intermediate orientation between the unconstrained configurations 670A and 670B. In some embodiments, the pushing and pulling of deployment from the delivery catheter 700 into the aneurysm 800, or other treatment site such as a blood vessel, may cause a change in orientation of the proximal section 650, for example a change in conformation from an unconstrained orientation wherein a proximal edge 670 faces away from, or is parallel to, the distal end 610 to an orientation wherein the proximal edge 670 faces toward, or parallel to, the distal end 610. Deployment of a proximal region comprising a flange or protuberance, such as the proximal region 650 of device 600, from a radial constrained, axially lengthened configuration within a catheter 700 to an expanded configuration near the neck region 830 of the aneurysm 800 may advantageously increase the number of layers of material at or near the neck of the aneurysm.

In some embodiments, the implantable device 600 may comprise a resilient, single-layer braided shell having an open distal end 610 and a closed proximal gathering section 680. The implantable device may additionally comprise a proximal region 650 comprising an upper layer of material 665, a lower layer of material forming a proximal surface 660, and a proximal edge 670 wherein a region near the proximal edge may comprise a radius of curvature or an inflection zone forming an angle or gap between the upper layer of material 665 and the lower layer or surface 660. To form a deployment configuration, the device may be radially compressed and axially lengthened within a catheter 700 wherein inflection zone may also be lengthened and the gap between the upper and lower proximal layers lengthens. While in the deployment configuration, the device may comprise a single layer of resilient mesh detachably coupled to a delivery catheter and capable of being pushed through the catheter to a treatment site. During deployment, the proximal region may form a double-layer proximal region comprising the upper layer of material 665 and the lower layer of material 660. In some embodiments, the proximal region may form a triple-layer of material comprising an intermediate layer 640, the upper layer 665, and the lower layer 660. In some embodiments, the treatment site may comprise a blood vessel and the double-layer or triple-layer proximal region may form a plug or obstruction to reduce blood flow within the blood vessel. In some embodiments, the treatment site may comprise an aneurysm and the double-layer or triple layer proximal region may be formed at the neck of the aneurysm. In some embodiments, the double-layer or triple-layer proximal region may be formed at the inflow and/or outflow areas at the neck of the aneurysm.

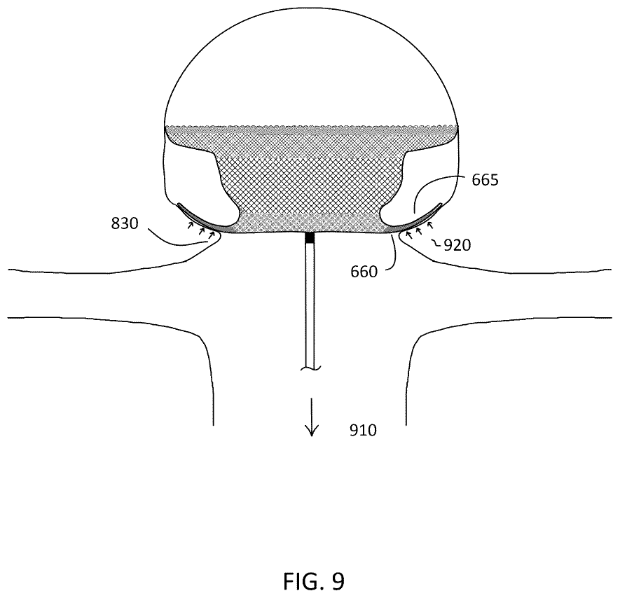

In some embodiments, a double layer or triple layer of resilient braided material may be formed from a single-layer implantable device at the neck of an aneurysm, or at the inflow and/or outflow of an aneurysm, by deploying an open-ended implantable device comprising an intermediate region 640, upper and lower proximal layers 665 and 660, and a proximal inflection zone. In some embodiments, the proximal upper 665 and lower proximal layer 660 may be compressed as shown in FIG. 9 by, for example, deploying the implantable device shown in FIG. 8 wherein the implantable device is detachably coupled to a delivery system, and placing a tension shown in arrow 910 on the delivery system. The tension 910 may create compressive forces shown by the arrows 920 on the lower proximal surface 660, reducing the unconstrained gap between the layers 665 and 660. Reduction of the gap between the upper and lower layers 665, 660 may have the beneficial effect of creating a higher pore density near the neck 830 of the aneurysm.

Some embodiments may include a method for treating a patient comprising: deploying within a blood vessel having vessel walls that is within the body of the patient, a device comprising (i) a resilient mesh structure formed from one or more filaments, the structure having a delivery shape and a deployed shape capable of conforming to the vessel walls; (ii) a single layer of resilient braided mesh having a substantially open distal end defining a first circumference and a substantially closed proximal end having a gathering section; (iii) a delivery system detachably coupled to the gathering section; (iv) a proximal section defining a second circumference wherein the second circumference is smaller than the first circumference; (v) a flange within the proximal section, the flange comprising an upper layer and a lower layer and a gap between the upper layer and the lower layer wherein the gap becomes smaller during deployment and forms multiple layers of resilient material near the proximal end of the device.

Some embodiments may include a method for treating an aneurysm having a neck, the method comprising: deploying within the aneurysm, a device comprising (i) a resilient mesh structure formed from one or more filaments, the structure having a delivery shape and a deployed shape capable of conforming to the aneurysm walls; (ii) a single layer of resilient braided mesh having a substantially open distal end defining a first circumference and a substantially closed proximal end having a gathering section; (iii) a delivery system detachably coupled to the gathering section; (iv) a proximal section defining a second circumference wherein the second circumference is smaller than the first circumference; and (v) a protuberance within the proximal section, the protuberance comprising an upper layer and a lower layer and a gap between the upper layer and the lower layer wherein the gap becomes smaller during deployment.

Some embodiments may include a method for treating an aneurysm having an equatorial region and a neck region, the method comprising: deploying within the aneurysm, a device comprising (i) a resilient mesh structure formed from one or more filaments, the structure having a delivery shape and a deployed shape capable of conforming to the aneurysm walls; (ii) a single layer of resilient braided mesh having a substantially open distal end defining a first circumference and a substantially closed proximal end having a gathering section; (iii) a delivery system detachably coupled to the gathering section; (iv) a proximal section defining a second circumference wherein the second circumference is configured to substantially conform the neck region of the aneurysm; (v) a flange within the proximal section, the flange comprising an upper layer and a lower layer and a gap between the upper layer and the lower layer; the method further comprising the steps of (a) deploying the distal end of the device near the equatorial region of the aneurysm; (b) deploying the flange of the device near the neck region of the aneurysm; (c) placing tension on the delivery system to reduce the gap between the upper layer and lower layer of the flange.

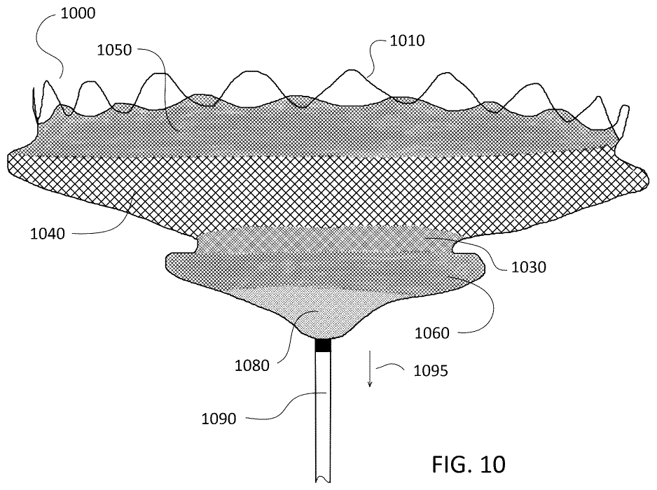

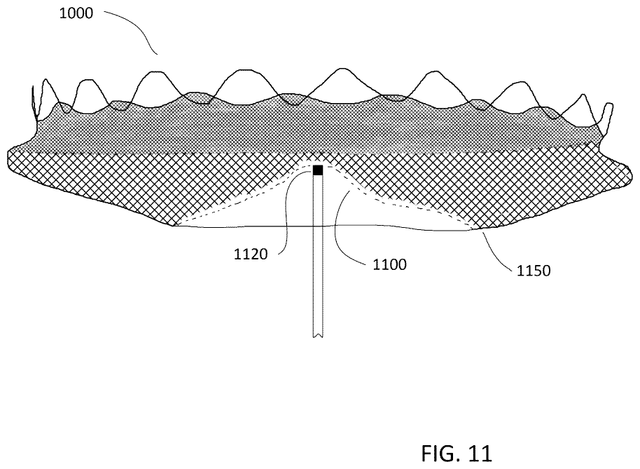

As shown in FIG. 10-11, in some embodiments, a device 1000 for occluding a blood vessel or aneurysm may comprise a single layer or multi-layer braided shell having a distal looped end section 1010. The looped end section 1010 may be formed from a series of filaments looped over a castellated braiding mandrel. The filaments may then be braided for approximately 1-30 wire crossings to form the distal looped end section 1010 before transitioning to a more densely braided distal section 1050. The braided shell may comprise multiple sections including the distal section 1050, a first intermediate section 1040, a second intermediate section 1030, a flanged section 1060, and/or a proximal section 1080. In some embodiments, there may be additional transitional sections between the named sections. The distal section 1050 may comprise a braided shell having a first pore density D1, a first pore circumferential length C1, and/or a first pore longitudinal length L1. The first intermediate section 1040 may comprise a braided shell having a second pore density D2, a second pore circumferential length C2, and/or a second pore longitudinal length L2. In some embodiments, D1 is greater than D2. In some embodiments, D1 is 10%-30% greater than D2. In some embodiments, D1 is 31%-100% greater than D2. In some embodiments, D1 is 101%-300% greater than D2. In some embodiments, D1 is over 300% greater than D2. In some embodiments, the distal section 1050 comprises at least one pore having a circumferential length D1 that is longer than the circumferential length D2 of at least one pore in the first proximally adjacent intermediate section 1040. In some embodiments, the distal section 1050 comprises at least one pore having a longitudinal length L1 that is longer than the longitudinal length L2 of at least one pore in the first proximally adjacent intermediate section 1040. In some embodiments, the distal section 1050 may be configured to be placed approximately near the equatorial region of an aneurysm. In some embodiments, the distal section 1050 may comprise a braided shell having at least one open end, the braided shell forming pores around a circumference, each pore having a circumferential length C1 wherein the circumferential length C1 is approximately equal to the circumferentially adjacent pore and wherein the distal section 1050 comprises at least one circumferential ring of pores, the ring approximately centered along a central axis Z. In some embodiments, the distal section may comprise 1-1000 rings of circumferentially arrayed pores. In some embodiments, the distal section may comprise 10-100 rings of circumferentially arrayed pores. In some embodiments, the distal section may comprise 101-200 rings of circumferentially arrayed pores. In some embodiments, the distal section may comprise 200-500 rings of circumferentially arrayed pores. In some embodiments, the distal section may comprise 501-1000 rings of circumferentially arrayed pores. In some embodiments, the distal section may comprise more than 1000 rings of circumferentially arrayed pores.

In some embodiments, the first intermediate section 1040 may form a tapered structure comprised of multiple circumferential rings of pores, at least one ring approximately centered along a central axis Z. The rings may reduce diameter along the central axis such that a first ring nearer to the distal section 1050 has a larger diameter than a second ring nearer to the proximal section 1080. In some embodiments, the tapered structure may comprise an angle measurable from the central axis Z to a plane defined by the outer edge of the intermediate section 1040. In some embodiments, the angle of the taper may be less than 90 degrees. In some embodiments, the angle of the taper may be less than 60 degrees. In some embodiments, the angle of the taper may be less than 45 degrees. In some embodiments, the angle of the taper may be less than 30 degrees. In some embodiments, the angle of the taper may be between 10 degrees and 30 degrees. In some embodiments, the angle of the taper may be between 5 degrees and 20 degrees.

In some embodiments, the second intermediate section 1030 is proximal to the first intermediate region 1040. The second intermediate section 1030 may comprise a braided shell having a pore density D3. In some embodiments, pore density D3 may be larger than the pore density of a distally adjacent intermediate section. In some embodiments, D3 may be larger than the pore density D2 of the first intermediate section 1040. In some embodiments, D3 is 10%-30% greater than D2. In some embodiments, D3 is 31%-100% greater than D2. In some embodiments, D3 is 101%-300% greater than D2. In some embodiments, D3 is over 300% greater than D2.

In some embodiments, a proximal flange section 1060 is proximal to the first and/or second intermediate sections. In some embodiments, the flange section 1060 may define a diameter larger than the diameter of the distally adjacent intermediate section 1030. The flange section 1060 may comprise a braided shell having a pore density D4. In some embodiments, pore density D4 may be larger than the pore density D2 of the first intermediate section 1040. In some embodiments, D4 may be larger than the pore density D2 of the first intermediate section 1040. In some embodiments, D4 is 10%-30% greater than D2. In some embodiments, D4 is 31%-100% greater than D2. In some embodiments, D4 is 101%-300% greater than D2. In some embodiments, D4 is over 300% greater than D2. The flange section 1060 may have more than one configuration. In a first configuration shown in FIG. 10, the device 1000 is detachably connected to a delivery system 1090. During deployment within an aneurysm or blood vessel, tension 1095 in the delivery system 1090 pulls the flange section 1060 and/or the proximal section 1080 into the first configuration. In some embodiments, in the first configuration, a proximal marker or gathering section 1120 may be located outside of the proximal section 1080. In a second configuration shown in FIG. 11, the proximal marker 1120 may be located inside the braided shell structure of the device 1000. For clarity in FIG. 11, sections of the braided shell are not shown and the proximal marker may be located at any point within braided shell structure of device 1000 when the device 1000 is in the unconstrained state, when the tension 1095 is relieved, when the delivery system pushes the gathering section 1120 into location, when the device is detached from the delivery system, or when a combination of these conditions occur.

In some embodiments, the flange section 1060 comprises a braided shell having an upper layer and a lower layer. The upper and lower layers may be configured to be approximately in contact with each other in an unconstrained state. In some embodiments, the upper and lower layers may be separated by a gap distance, forming a device with at least two layers at the flange section 1060. In some embodiments, the flange section may be perpendicular to a central axis Z. In some embodiments, the flange may form a circumferential outer edge wherein the edge is biased away from the distal end section 1010. In some embodiments, in an un-tensioned state, a portion of the proximal section 1100 may be within one or more intermediate sections 1040 and/or 1030. In some embodiments, in an unconstrained state, the device 1000 may define a proximal perimeter 1150 and the proximal gathering section 1120 may be distal to the proximal perimeter. In some embodiments, the proximal section 1100 may be distal to the proximal perimeter 1150 in an un-tensioned state.

In some embodiments, the flange section 1060 may be configured for placement at the neck of an aneurysm. In some embodiments, the implantable device 1000 may be detachably coupled to the delivery system 1090. Some embodiments may include a method for treating an aneurysm having an equatorial region and a neck region, the method comprising: deploying within the aneurysm, a device comprising (i) a resilient mesh structure formed from one or more filaments, the structure having a delivery shape and a deployed shape capable of conforming to the aneurysm walls; (ii) a single layer of resilient braided mesh having a substantially open distal end defining a first circumference and a substantially closed proximal end having a gathering section; (iii) a delivery system detachably coupled to the gathering section; (iv) a flange section defining a second circumference wherein the second circumference is configured to substantially conform the neck region of the aneurysm wherein the flange comprises an upper layer and a lower layer and a gap between the upper layer and the lower layer; the method further comprising the steps of (a) deploying the distal end of the device near the equatorial region of the aneurysm; (b) deploying the flange of the device near the neck region of the aneurysm; (c) placing tension on the delivery system to decrease the gap between the upper layer and lower layer of the flange; and (d) detaching the gathering section from the delivery system.

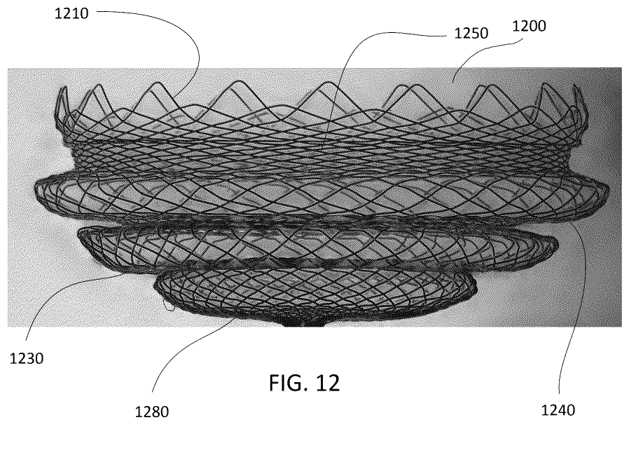

FIG. 12 illustrates an embodiment of a single layer braided device 1200 having an open distal end comprising castellated loops 1210 and a closed proximal end, wherein the device comprises multiple tiers 1250, 1240, 1230, and 1280. In some embodiments, each tier may comprise a specific braid pattern and each tier defines a diameter. The distal tier 1250 may comprise a braid comprising pores, wherein each pore has a longitudinal length and a circumferential length and wherein the circumferential length of at least one pore in the distal tier is longer than longitudinal length of the same pore. In some embodiments, the pore density of tier the distal tier 1250 may be higher than the pore density of the proximally adjacent tier 1240. Tier 1240 may be positioned proximally adjacent to tier 1250. In some embodiments, the diameter of tier 1240 may be larger than the diameter of tier 1250. The braid pattern of tier 1240 may be less dense than the distally adjacent tier 1250. Tier 1240 may comprise pore(s) having a longitudinal length and a circumferential length, wherein the longitudinal length of at least one pore is approximately equal to the circumferential length of the same pore. In some embodiments, the circumferential length of at least one pore may be longer than the longitudinal length of the same pore. The device 1200 may comprise another tier 1230 proximally adjacent to tier 1240 wherein the diameter of tier 1230 may be equal to or less than the diameter of tier 1240. The device 1200 may comprise a proximal tier 1280 wherein the proximal tier may have a pore density greater than the distally adjacent tier 1230. The proximal tier 1280 may comprise pore(s) wherein each pore has a longitudinal length and a circumferential length wherein at least one pore has a circumferential length longer than the longitudinal length of the same pore. The device 1200 may be configured to have an unconstrained configuration exemplified by FIG. 12 and a constrained configuration adapted for delivery through a microcatheter. In some embodiments, the device 1200 comprises a single-layer braided shell construction wherein the braided shell does not fold over itself when in the constrained deployment configuration. In some embodiments, the device 1200 may be deployed in an aneurysm having a dome and a neck wherein the dome and the neck of the aneurysm each define a diameter. In some embodiments, the device 1200 may be configured so that the diameter of tier 1240 is approximately 5% to 50% larger than the diameter of the aneurysm dome. In some embodiments, the device 1200 may be configured so that the diameter of tier 1240 is approximately 10% to 40% larger than the diameter of the aneurysm dome. In some embodiments, the device 1200 may be configured so that the diameter of tier 1240 is approximately 0.5 mm to 3.0 mm larger than the diameter of the aneurysm dome. In some embodiments, the device 1200 may be configured so that the diameter of tier 1280 is approximately 0% to 50% larger than the diameter of the aneurysm neck. In some embodiments, the device 1200 may be configured so that the diameter of tier 1280 is approximately 5% to 40% larger than the diameter of the aneurysm dome. In some embodiments, the device 1200 may be configured so that the diameter of tier 1280 is approximately 0.0 mm to 3.0 mm larger than the diameter of the aneurysm dome. In some embodiments, the proximal tier 1280 may be configured to compress, fold, or be positioned at the neck of the aneurysm during deployment. In some embodiments compressing, folding, or positioning tier 1280 at or near the neck of the aneurysm during deployment may form multiple layers of braid from a single-layer device, the layers of braid comprising the proximal edge of the proximal tier 1280 and the distal edge of the proximal tier 1280 wherein the distal edge of the proximal tier 1280 may be proximally adjacent to the distal edge of tier 1230. In some embodiments, the proximal tier 1280 is formed from a single layer of braid in the unconstrained configuration and may be configured to compress or fold at or near the neck of the aneurysm during deployment and, in some embodiments, may form a multi-layer braid during deployment. In some embodiments compressing, folding, or positioning tier 1280 at the neck of the aneurysm may form multiple layers of braid comprising a proximal portion of the proximal tier 1280 and a distal portion of the proximal tier 1280. In some embodiments compressing, folding, or positioning tier 1280 at the neck of the aneurysm may form multiple layers of braid comprising a proximal portion of the proximal tier 1280 and a distal portion of the proximal tier 1280, and a proximal portion of an intermediate tier 1230. In some embodiments, the device 1200 comprises a single layer braided shell having an open distal end and a closed proximal end, wherein the device is configured to form one, two, three, or more layers of braided material at or near the neck of an aneurysm when the device is deployed from a microcatheter positioned within the aneurysm or near the neck of an aneurysm. In some embodiments, the aneurysm may comprise an inflow zone and/or an outflow zone. In some embodiments, the device 1200 comprises a single layer braided shell having an open distal end and a closed proximal end, wherein the device is configured to form one, two, three, or more layers of braided material when deployed at or near the neck of an aneurysm and may occlude, slow, or obstructing blood flow into the aneurysm.

In some embodiments, the proximal tier 1280 may be configured to compress, fold, or be positioned in a blood vessel during deployment from a compressed configuration within a catheter or microcatheter. In some embodiments, the proximal tier 1280 is formed from a single layer of braid in the unconstrained configuration and may be configured to compress or fold at within the blood vessel during deployment and, in some embodiments, may form a multi-layer braid during deployment. In some embodiments compressing, folding, or positioning tier 1280 in the blood vessel may form multiple layers of braid comprising a proximal portion of the proximal tier 1280 and a distal portion of the proximal tier 1280. In some embodiments compressing, folding, or positioning tier 1280 in the blood vessel may form multiple layers of braid comprising a proximal portion of the proximal tier 1280 and a distal portion of the proximal tier 1280, and a proximal portion of an intermediate tier 1230. In some embodiments, the device 1200 comprises a single layer braided shell having an open distal end and a closed proximal end, wherein the device is configured to form one, two, three, or more layers of braided material within the blood vessel when the device is deployed from a catheter. In some embodiments, the device 1200 comprises a single layer braided shell having an open distal end and a closed proximal end, wherein the device is configured to form one, two, three, or more layers of braided material when deployed within a blood vessel and may occlude, slow, or obstructing blood flow in the blood vessel.

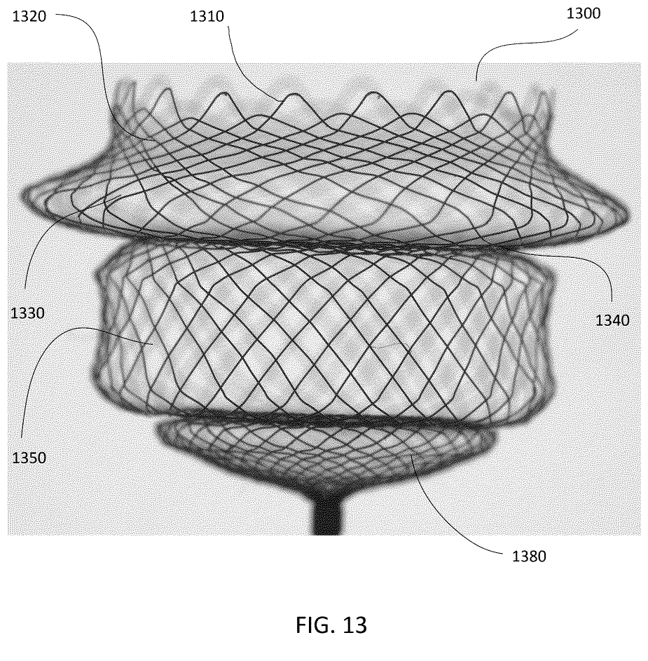

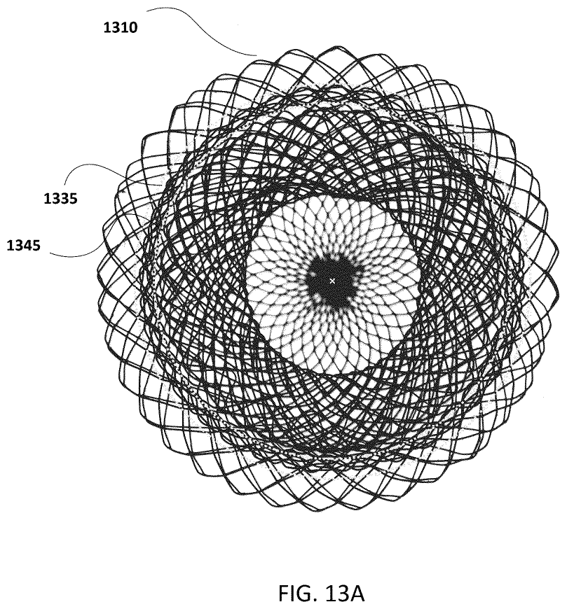

FIG. 13 shows an embodiment of a single layer braided implantable device 1300 having an open distal end, a closed proximal end, and a total length. The open end may comprise of a series of castellated loops 1310 and a distal section 1320 wherein the distal section has a diameter and may comprise one or more pore(s), each pore having a longitudinal length and a circumferential length wherein at least one pore in the distal section has a circumferential length longer than the longitudinal length of the same pore. The device 1300 may comprise a first intermediate section 1330, approximately shaped like a saucer or disc, having a diameter, wherein the diameter of the first intermediate section 1330 is larger than the diameter of the distal section 1320. The device 1300 may comprise second intermediate section 1350, approximately shaped like a cylinder, having a diameter at an inflection section 1340, a length, and comprising pore(s). In some embodiments, the intermediate cylinder-shaped section 1350 comprises an unconstrained length. In some embodiments, the unconstrained length of section 1350 is approximately 20% to 70% of the total length of the device 1300. In some embodiments, the unconstrained length of section 1350 is approximately 10% to 40% of the total length of the device 1300. In some embodiments, the unconstrained length of section 1350 is approximately 20% to 30% of the total length of the device 1300. In some embodiments, the length of the second intermediate section 1350 is approximately 10%-60% of the total length of the device 1300 in an unconstrained state. In some embodiments, the length of the second intermediate section 1350 is approximately 25%-35% of the total length of the device 1300 in an unconstrained state. In some embodiments, the length of the second intermediate section 1350 is approximately 13%-33% of the total length of the device 1300 in an unconstrained state. In some embodiments, the length of the second intermediate section 1350 is approximately 10%-25% of the total length of the device 1300 in an unconstrained state. In some embodiments, the diameter of the second intermediate section 1350 may be equal to or less than the diameter of the of the first intermediate section 1330. In some embodiments, the intermediate section 1330 may overlap the adjacent intermediate section 1350, the overlap section being formed approximately near the inflection point 1340. FIG. 13A is a top view of a single layer device having an open distal end and a closed proximal end, the device comprising distal loops 1310, and an overlap section approximately near an inflection between two sections of the device between the distal end and the proximal end, shown in FIG. 13A by an outer dotted line 1335 and an inner dotted line 1345. In some embodiments, the overlap section has a length or overlap distance approximately equal to the distance between the inner dotted line 1345 and the outer dotted line 1335. In some embodiments, the device 1300 comprises a first intermediate section 1330 and a proximally adjacent second intermediate section 1350 wherein the first intermediate section defines a first diameter, and the second intermediate section defines a second diameter. In some embodiments, the first diameter and second diameter overlap wherein the overlap distance is approximately equal to the absolute value of the difference between the first diameter and the second diameter. In some embodiments, the overlap distance is 0.1 mm to 10 mm. In some embodiments, the overlap distance is 0.1 mm to 3 mm. In some embodiments, the overlap distance is 0.5 mm to 5 mm. In some embodiments, the overlap distance is 1 mm to 10 mm. In some embodiments, the overlap distance is 0.25 mm to 3 mm. In some embodiments, the overlap distance is 1% to 30% of the first diameter of the distal intermediate section. In some embodiments, the overlap distance is 1% to 5% of the first diameter of the distal intermediate section. In some embodiments, the overlap distance is 1% to 7% of the first diameter of the distal intermediate section. In some embodiments, the overlap distance is 1% to 5% of the first diameter of the distal intermediate section.

In some embodiments, the pore(s) of the second intermediate section 1350 may comprise a longitudinal length and a circumferential length wherein a least one pore in the second intermediate section 1350 has longitudinal length equal to or greater than the circumferential length of the same pore. The device 1300 may comprise a flattened, conical, or frustoconical shaped proximal tier 1380 wherein the proximal tier may have a pore density greater than the distally adjacent tier 1350. The proximal tier 1380 may comprise pore(s) wherein each pore has a longitudinal length and a circumferential length wherein at least one pore has a circumferential length longer than the longitudinal length of the same pore.

The device 1300 may be configured to have an unconstrained configuration and a constrained configuration adapted for delivery through a microcatheter. In some embodiments, the device 1300 comprises a single-layer braided shell construction wherein the braided shell does not fold over itself when in the constrained deployment configuration. In some embodiments, the device 1300 comprises a single-layer braided shell construction wherein the braided shell does not fold over itself when in an unconstrained configuration. In some embodiments, the device 1300 may be deployed in an aneurysm having a dome and a neck wherein the dome and the neck of the aneurysm each define a diameter. The device 1300 may be configured so that the diameter of section 1330 is approximately 5% to 50% larger than the diameter of the aneurysm dome. In some embodiments, the device 1300 may be configured so that the diameter of an intermediate section near the distal section is approximately 10% to 40% larger than the diameter of the aneurysm dome. In some embodiments, the device 1300 may be configured so that the diameter of a saucer or disc-shaped section 1330 is approximately 0.5 mm to 3.0 mm larger than the diameter of the aneurysm dome. In some embodiments, the device 1300 may be configured so that the diameter of the proximal tier 1380 is approximately 0% to 50% larger than the diameter of the aneurysm neck. In some embodiments, the device 1300 may be configured so that the diameter of the saucer or disc-shaped section 1330 is approximately 5% to 40% larger than the diameter of the aneurysm dome. In some embodiments, the device 1300 may be configured so that the diameter of an intermediate section 1330 is approximately 0.1 mm to 3.0 mm larger than the diameter of the aneurysm dome. In some embodiments, the diameter of section 1330 may be approximately 0% to 10% larger than the diameter at the inflection section 1340 of the cylindrical section 1350. In some embodiments, the diameter of section 1330 may be approximately 5% to 15% larger than the diameter of the inflection section 1340. In some embodiments, the diameter of section 1330 may be approximately 15% to 25% larger than the diameter of the cylindrical section 1350. In some embodiments, the diameter of section 1330 may be approximately 0% to 30% larger than the diameter of the inflection section 1340. In some embodiments, the diameter of section 1330 may be approximately 3% to 9% larger than the diameter of the inflection section 1340.

In some embodiments, the proximal tier 1380 may be configured to compress, fold, or be positioned at the neck of the aneurysm during deployment. In some embodiments compressing, folding, or positioning tier 1380 at or near the neck of the aneurysm during deployment may form multiple layers of braid from a single-layer braided device, the layers of braid comprising the proximal edge of the proximal tier 1380 and the distal edge of the proximal tier 1380 wherein the distal edge of the proximal tier 1380 may be proximally adjacent to the distal edge of tier 1350. In some embodiments, the proximal tier 1380 is formed from a single layer of braid in the unconstrained configuration and may be configured to compress or fold at or near the neck of the aneurysm during deployment and, in some embodiments, may form a multi-layer braid during deployment. In some embodiments compressing, folding, or positioning tier 1380 at the neck of the aneurysm may form multiple layers of braid comprising a proximal portion of the proximal tier 1380 and a distal portion of the proximal tier 1350. In some embodiments compressing, folding, or positioning tier 1380 at the neck of the aneurysm may form multiple layers of braid comprising a proximal portion of the proximal tier 1380 and a distal portion of the proximal tier 1380, and a proximal portion of an intermediate tier such as the cylindrical section 1350. In some embodiments, the device 1300 comprises a single layer braided shell having an open distal end and a closed proximal end, wherein the device is configured to form one, two, three, or more layers of braided material at or near the neck of an aneurysm when the device is deployed from a microcatheter positioned within the aneurysm or near the neck of an aneurysm. In some embodiments, the aneurysm may comprise an inflow zone and/or an outflow zone. In some embodiments, the device 1300 comprises a single layer braided shell having an open distal end and a closed proximal end, wherein the device is configured to form one, two, three, or more layers of braided material when deployed at or near the neck of an aneurysm and may occlude, slow, or obstructing blood flow into the aneurysm.

FIG. 14 shows an embodiment of a device, for example, the device 1300 shown in FIG. 13, deployed in a simulated glass aneurysm 1425 having a neck 1445. The simulated aneurysm, like some human aneurysms, has a non-spherical geometry in which the height of the aneurysm is less than the diameter of the aneurysm. In some embodiments, the device is configured to fold or invert during deployment wherein, for example, an intermediate section 1330 is folded during deployment and at least a portion of an intermediate section overlaps at least a portion of a second intermediate section 1350. As shown in FIG. 14, the device comprises a braid having an open castellated end 1310. In the folded or inverted configuration, the castellated ends are oriented towards the top of the aneurysm. In the folded or inverted configuration, the edge of the intermediate section 1330, previously oriented approximately perpendicular to the center axis Z of the device in the unrestrained configuration, folds or inverts to an orientation wherein at least a portion of the braid forming the intermediate section is pointing towards the neck of the aneurysm 1445. In some embodiments, inverting or folding a single layer braided device during deployment into an aneurysm may form a multilayer braid within the aneurysm or within a blood vessel. In some embodiments, inverting or folding a single layer braided device during deployment into an aneurysm may form a multilayer braid within the aneurysm or within a blood vessel may increase the radial strength of the device at or near a distal portion of the device compared to the radial strength of a distal portion of the device in the unconstrained configuration. Advantageously, this may increase the radial pressure of the device against the wall of the aneurysm or blood vessel, improving the stability of the device and resisting movement caused by blood flow.

FIG. 14 shows a folded, multilayered configuration of the proximal section 1380 of the device 1300 when deployed into the aneurysm 1425 near the neck 1445. In some embodiments, the proximal section 1380 is a single layer of braid in an unrestrained state and may be configured to fold or compress during deployment within a blood vessel or aneurysm and may form a multilayer braided proximal section when deployed. Advantageously, the multilayer braided proximal section positioned near the neck of an aneurysm may block or obstruct the inflow or outflow of blood into the aneurysm. Advantageously, the multilayer braided proximal section positioned within a blood vessel may block or obstruct the blood flow within the blood vessel.

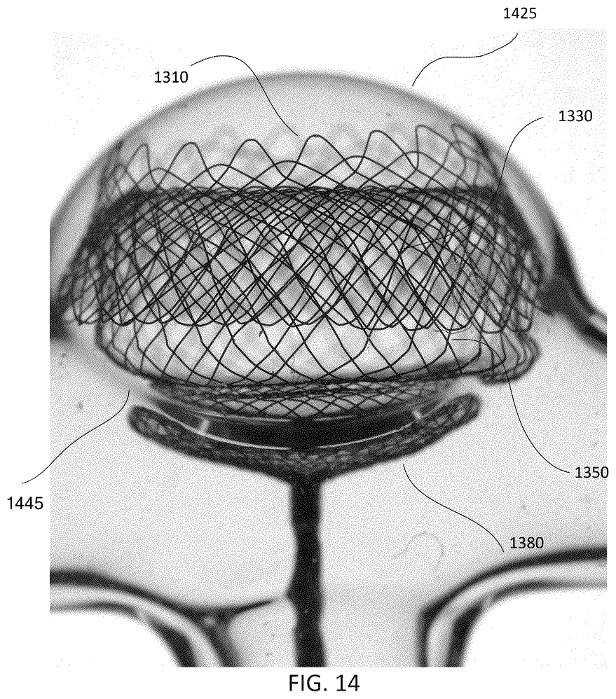

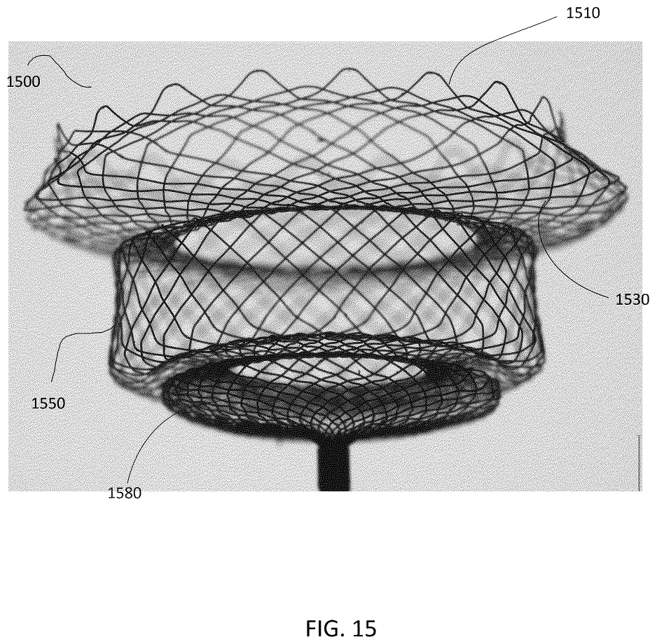

FIG. 15 shows an embodiment of a single-layer braided device 1500 comprising an open, castellated end 1510 comprising looped wires, a flattened disc or saucer shaped distal section 1530 having a pore density, a cylindrical intermediate section 1550 having a pore density, and a closed proximal section 1580 having a pore density wherein one or more wires comprising the device are gathered in, for example, an approximately cylindrical metallic or polymer band. In some embodiments, an angle may be formed between the distal section 1530 and proximally adjacent section 1550. In some embodiments, the angle formed is approximately 30 degrees to 140 degrees. In some embodiments, the angle formed is approximately 45 degrees to 100 degrees. In some embodiments, the angle formed is approximately 75 degrees to 100 degrees. In some embodiments, the angle formed is approximately 80 degrees to 95 degrees. In some embodiments, the distal section 1530 may define a first diameter. In some embodiments, the intermediate section 1550 proximally adjacent to the distal section may define a second diameter. In some embodiments, the first diameter may be larger than the second diameter. In some embodiments, the first diameter may be approximately equal to the second diameter. In some embodiments, the first diameter may be 0.5 mm to 5.0 mm larger than the second diameter. In some embodiments, the first diameter may be 10% to 50% larger than the second diameter. In some embodiments, the first diameter may be 20%-30% larger than the second diameter. In some embodiments, the first diameter may be 10%-20% larger than the second diameter.

In some embodiments, the saucer-shaped distal section 1530 may overlap the cylindrical shaped section 1550. This type of overlap is also illustrated by FIG. 13A. In some embodiments, the overlap defines a double layer or multilayer overlap section having a pore density, wherein the pore density of the overlap section is more dense or greater than the pore density of the distal section 1530. In some embodiments, the pore density of the overlap section is more dense or greater than the pore density of the intermediate section 1550. In some embodiments, the pore density of the overlap section is more dense or greater than the pore density of either of the adjacent sections that form the overlap. In some embodiments, the braided filaments or wires forming the overlap section are not parallel with each other since the distal intermediate section is formed at a different braid angle from the proximal intermediate section. In an unexpected result, this makes the overlap pore density higher than would be expected from folding one layer on top of another. Thus, in some embodiments, the pore density of the overlap section is greater than the average of the pore densities of the first and second sections that form the overlap.

In some embodiments, the device 1500 comprises a proximal section 1580 having a length and a diameter. In some embodiments, the pore density of the proximal section 1580 is higher than the pore density of the adjacent section of the device. In some embodiments, the pore density of the distal section 1580 is higher than the pore density of at least one of the other sections of the device. In some embodiments, the pore density of the proximal section 1580 is higher than an approximately cylindrical intermediate section 1550 of the device 1500. In some embodiments, the pore density of the proximal section 1580 is the highest of any other section (for example, distal section 1530 or intermediate section 1550) of the device 1500. In some embodiments, the braided filaments or wires forming the sections 1550 and 1580 are not parallel with each other since two sections are formed at different braid angles. This makes the overlap pore density higher than would be expected from folding one layer on top of another. Thus, in some embodiments, the pore density of a section formed by overlapping or multilayered portions of the intermediate section 1550 and the proximal section 1580 are greater than the average of the pore densities of the sections forming the overlapping or multilayered section.

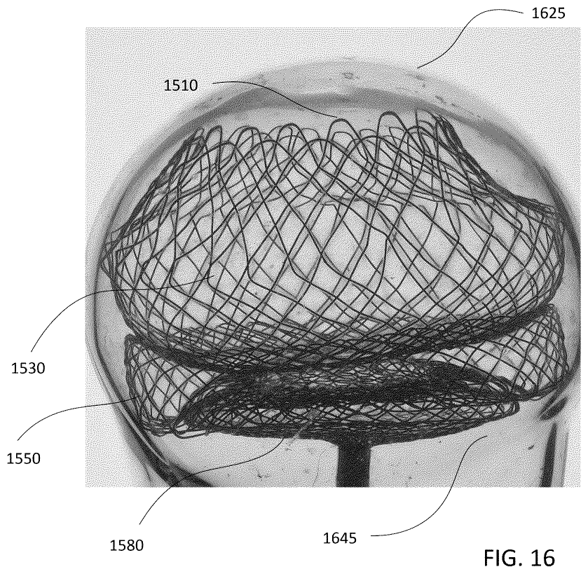

FIG. 16 shows an embodiment of the device 1500 deployed within a simulated glass aneurysm 1625 having a neck 1645. In the example shown in FIG. 16, the aneurysm is approximately spherical and has an equatorial diameter and a neck diameter. In some embodiments, the distal loops 1510 of the device 1500 are deployed near the dome of the aneurysm 1625. The diameter of the distal section 1530 may be 0.25 to 4 mm larger, or 10% to 40%, larger than the equatorial diameter of the aneurysm. In some embodiments, oversizing at least a portion of the distal section 1530 by, for example, 0.25 to 4 mm and/or 10% to 40%; may compress distal section 1530 and may cause approximately axial compression of the intermediate section 1550. In some embodiments, during or after deployment within an aneurysm, blood vessel, or malformation; the intermediate section 1550 may be configured to compress along an axis approximately parallel to the centerline Z of the device. In some embodiments, the axial length of intermediate section 1550 may be configured to compress approximately 10% to 75% during or after deployment in a blood vessel, aneurysm, or malformation compared to the axial length in the free-air or unconstrained configuration.

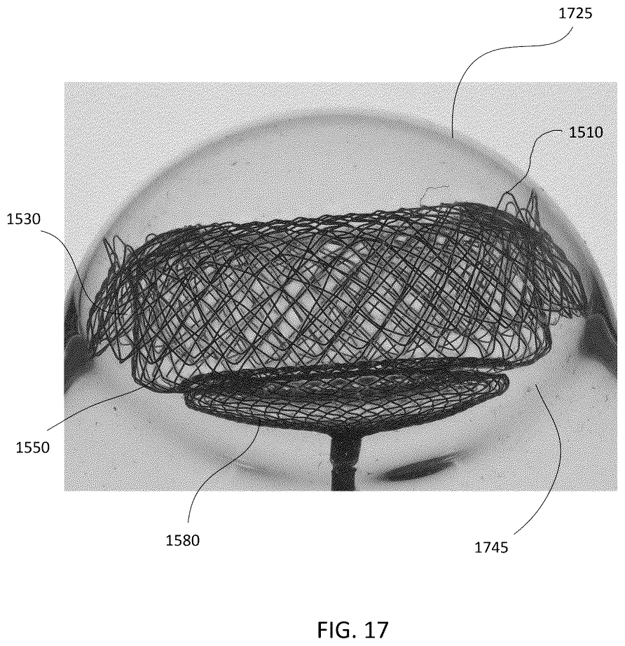

FIG. 17 shows an embodiment of a device, for example, the device 1500 shown in FIG. 15, deployed in a simulated glass aneurysm 1725 having a neck 1745. The simulated aneurysm, like some human aneurysms, has a non-spherical geometry in which the height of the aneurysm is less than the diameter of the aneurysm. In some embodiments, the device is configured to fold or invert during deployment wherein, for example, an intermediate section 1530 is folded during deployment and at least a portion of an intermediate section overlaps at least a portion of a second intermediate section 1550. As shown in FIG. 15, the device comprises a braid having an open castellated end 1510. In the folded or inverted configuration, the castellated ends are oriented towards the top of the aneurysm. In the folded or inverted configuration, the edge of the intermediate section 1530, previously oriented approximately perpendicular to the center axis Z of the device in the unrestrained configuration, folds or inverts to an orientation wherein at least a portion of the braid forming the intermediate section is pointing towards the neck of the aneurysm 1745. In some embodiments, inverting or folding a single layer braided device during deployment into an aneurysm may form a multilayer braid within the aneurysm or within a blood vessel. In some embodiments, inverting or folding a single layer braided device during deployment into an aneurysm may form a multilayer braid within the aneurysm or within a blood vessel may increase the radial strength of the device at or near a distal portion of the device compared to the radial strength of a distal portion of the device in the unconstrained configuration. Advantageously, this may increase the radial pressure of the device against the wall of the aneurysm or blood vessel, improving the stability of the device and resisting movement caused by blood flow.

In some embodiments, the sections of the device are configured to fold or not to fold, or compress or not to compress, depending on the geometry of the aneurysm or treatment site. In some embodiments the same device (for example, device 1300 or 1500) may be configured to have two or more stable configurations when deployed within different-shaped aneurysms. In a first instance, the device may be configured to deploy in an approximately spherical aneurysm having an equatorial section and a neck section, wherein the distal section may be configured to conform to the equatorial section of the aneurysm without flipping, a proximal section may be configured to conform to the neck section of the aneurysm, and an intermediate section may be configured to compress from a first unconstrained length to a second constrained length. In a second instance, the device may be configured to deploy in an aneurysm having an equatorial diameter, a neck, and a height, wherein the height may be greater than the equatorial diameter and wherein the distal section may be configured to conform to the equatorial section of the aneurysm without flipping, a proximal section may be configured to conform to the neck section of the aneurysm, and an intermediate section may be configured to compress from a first unconstrained length to a second constrained length wherein the second constrained length when deployed in an aneurysm having a height greater than the equatorial diameter is less than the second constrained length of the intermediate section when deployed in an approximately spherical aneurysm. In a third instance, the device may be configured to deploy in an aneurysm having an equatorial diameter, a neck section, and a height wherein the height may be less than the equatorial diameter and wherein a distal section of the device may be configured to at least partially flip over an intermediate section of the device, and wherein a proximal section of the device may be configured to conform to the neck section of the aneurysm. Advantageously and unexpectedly, in some embodiments, two or more of the three instances can be achieved with the same device conformation.

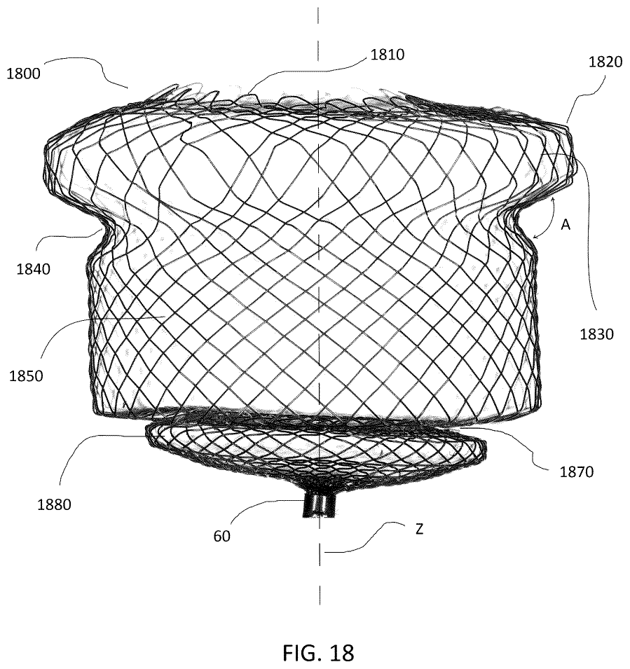

FIG. 18 shows an embodiment of a single layer braided implantable device 1800 in an unconstrained configuration, the device comprising an open distal end with castellated loops 1810, a distal edge 1820, a distal section 1830, an inflection curvature 1840, an intermediate section 1850, a proximal section 1880, an overlap section 1870, a closed proximal end 60, and an approximately vertical central axis Z. In some embodiments, the distal loops 1810 may be bent inward toward the central axis Z to form the distal edge 1820. In some embodiments, distal loops comprise apices and the apices may be at an angle to the central axis Z. In some embodiments, the angle between the apex of at least one loop may be 0 degrees (i.e. the loop or loops lie approximately perpendicular to the central axis Z) to 60 degrees. In some embodiments, the angle between the apex of at least one loop and the axis Z may be 0 degrees to 10 degrees. In some embodiments, the angle between the apex of at least one loop and the axis Z may be 0 degrees to 45 degrees. In some embodiments, the angle between the apex of at least one loop and the axis Z may be 0 degrees to 90 degrees. In some embodiments, the angle between the apex of at least one loop and the axis Z may be 1 degree to 20 degrees. In some embodiments, the distal section 1830 may comprise a disk or flattened disk shape in which the distal edge 1820 is bent around a rounded fixture at the inflection point 1840 to form an angle A. In some embodiments, angle A is 3 degrees to 70 degrees. In some embodiments, angle A is 5 degrees to 60 degrees. In some embodiments, angle A is 3 degrees to 30 degrees. In some embodiments, angle A is 15 degrees to 45 degrees. In some embodiments, angle A is 12 degrees to 35 degrees. In some embodiments, the distal section has a diameter and is configured for deployment within a treatment site such as an aneurysm or a blood vessel having a maximum diameter wherein the diameter of the distal section 1830 in the unconstrained state may be larger than the maximum diameter of the treatment site. In some configurations, the diameter of the distal section may be 0.5 mm to 5 mm larger than the maximum diameter of the treatment site. In some embodiments, the diameter of the distal section may be 5% to 50% larger than the maximum diameter of the treatment site. In some embodiments, the intermediate section 1850 may be approximately cylindrically shaped and may comprise a diameter and a length. In some embodiments, the unconstrained diameter of the intermediate section 1850 may be less than the unconstrained length of the intermediate section. In some embodiments, the unconstrained diameter of the intermediate section 1850 may be greater than or equal to the unconstrained length of the intermediate section.

In some embodiments, the device 1800 may be configured for deployment within a vascular defect such as a brain aneurysm, or peripheral aneurysm, or the left atrial appendage of a human heart, the vascular defect having an equatorial diameter, a neck, and a distance between the equatorial diameter and the neck, wherein the length of the intermediate section 1850, in a constrained state within the vascular defect, may be approximately equal to between 20% and 100% of the distance between the equatorial diameter of the vascular defect and the neck of the vascular defect. In some embodiments, the length of the intermediate section 1850, in a constrained state within the vascular defect, may be approximately equal to between 30% and 90% of the distance between the equatorial diameter of the vascular defect and the neck of the vascular defect. In some embodiments, the length of the intermediate section 1850, in a constrained state within the vascular defect, may be approximately equal to between 60% and 80% of the distance between the equatorial diameter of the vascular defect and the neck of the vascular defect. In some embodiments, the length of the intermediate section 1850, in a constrained state within the vascular defect, may be approximately equal to between 75% and 90% of the distance between the equatorial diameter of the vascular defect and the neck of the vascular defect.

In some embodiments, the proximal section 1880 may comprise a conical, frustoconical, or disc shape. In some embodiments, the diameter of the proximal section is less than the adjacent intermediate section. In some embodiments, the pore density of the proximal section may be greater than the pore density of the adjacent intermediate section. In some embodiments, the diameter of the proximal section is less than the adjacent intermediate section. In some embodiments, the pore density of the proximal section may be greater than the pore density of any portion of the device distal to the overlap section 1870. In some embodiments, the proximal section may be configured to deploy with multiple folded sections comprising a proximal layer of braid of the proximal section 1880 and a distal layer of braid adjacent to the overlap section 1870 of the proximal section 1880. In some embodiments, the proximal section may be configured to deploy with multiple folded sections comprising a proximal layer of braid of the proximal section 1880, a distal layer of braid adjacent to the overlap section 1870 of the proximal section 1880, and a portion of the intermediate section 1850. In some embodiments, the device comprises a closed proximal end 60. The closed proximal end 60 may be formed by gathering the braided wires or filaments of the device 1800 into a cylindrical band and, for example, bonding the filaments to the band by laser welding, crimping, adhesive bonding, epoxy bonding, or soldering.

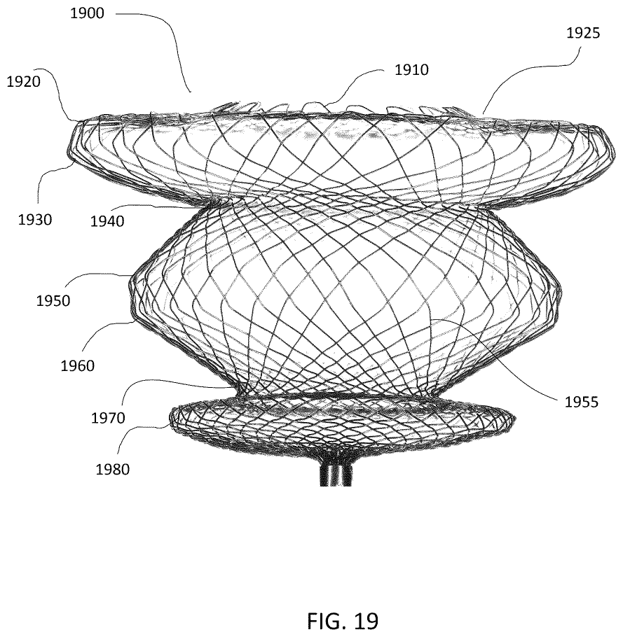

FIG. 19 shows an embodiment of a single layer braided device 1900, the device comprising an open castellated end comprising looped filaments 1910, a closed proximal end, a distal tier 1925 comprising a first edge 1920 and a second edge 1930, an intermediate tier 1955 comprising a first edge 1950 and a second edge 1960 and an inflection section 1940 between the distal tier 1925 and the intermediate tier 1955. The device 1900 may further comprise a proximal tier 1980 with a closed proximal end configured for placement within the neck of an aneurysm or within the lumen of a blood vessel. The device may comprise a proximal inflection section 1970 near the transition from the intermediate tier 1955 and the proximal tier 1980. The device 1900 comprises a total length in an unconstrained configuration, the total length of the device may be defined as the axial distance parallel or coincident with a central axis Z between the distal-most looped filament 1910 and the proximal-most portion of the closed end. In some embodiments, the intermediate section 1955 may be approximately spherical with a flattened top and bottom, or may comprise a trapezoid shape, or may comprise a shape approximating two trapezoids placed on top of each other with the long axis of each trapezoid on top of the other trapezoid.

In some embodiments, the distal loops 1910 comprise vertices or apices wherein the apices are approximately oriented perpendicular to the vertical axis Z of the device 1900. In some embodiments, the distal tier 1925 comprises the distal edge 1920 wherein the distal edge defines a first diameter, and the apices of the distal loops define a second diameter. In some embodiments, the second diameter defined by the loop apices is approximately 30% to 95% of the first diameter. In some embodiments, the second diameter is approximately 50% to 80% of the first diameter. In some embodiments, the second diameter is approximately 30% to 70% of the first diameter. In some embodiments, the second diameter is approximately 60% to 80% of the first diameter. In some embodiments, the second diameter is approximately 70% to 90% of the first diameter. The first edge 1920 and the second edge 1930 may define a length between the two edges. In some embodiments, the length between the two edges may be approximately 3% to 30% of the total length of the device. In some embodiments, the length between the two edges may be approximately 5% to 10% of the total length of the device. In some embodiments, the length between the two edges may be approximately 3% to 10% of the total length of the device. In some embodiments, the length between the two edges may be approximately 8% to 20% of the total length of the device.

The braided section between second edge 1930 and the inflection section 1940 may define a distal plane (or a distal cone when considering the device as a three dimensional structure), the distal plane or cone having a first tangency near the edge 1930 and a second tangency near the inflection section 1940. In some embodiments, the braided section between the inflection section 1940 and the first intermediate edge 1950 of the intermediate tier 1955 may define a first intermediate plane or cone having tangencies near the inflection section 1940 and near the first intermediate edge 1950. In some embodiments, the distal plane or cone and the first intermediate plane or cone may intersect near the inflection section 1940, thereby defining an angle between the planes or cones. In some embodiments, the angle between the distal plane and the first intermediate plane may be between 2 degrees to 80 degrees. In some embodiments, the angle between the distal plane and the first intermediate plane may be between 3 degrees to 45 degrees. In some embodiments, the angle between the distal plane and the first intermediate plane may be between 10 degrees to 45 degrees. In some embodiments, the angle between the distal plane and the first intermediate plane may be between 20 degrees to 60 degrees. The distance between the first intermediate edge 1950 and the second intermediate edge 1960 defines a cylinder, the cylinder having a length and a diameter. In some embodiments, the length of the cylinder defined by the intermediate edges 1950 and 1960 in the unconstrained configuration is approximately between 3% and 30% of the total length of the device in the unconstrained configuration. In some embodiments, the length of the cylinder defined by the intermediate edges 1950 and 1960 in the unconstrained configuration is approximately between 3% and 40% of the total length of the device in the unconstrained configuration. In some embodiments, the length of the cylinder defined by the intermediate edges 1950 and 1960 in the unconstrained configuration is approximately between 3% and 15% of the total length of the device in the unconstrained configuration. In some embodiments, the length of the cylinder defined by the intermediate edges 1950 and 1960 in the unconstrained configuration is approximately between 5% and 10% of the total length of the device in the unconstrained configuration. In some embodiments, the length of the cylinder defined by the intermediate edges 1950 and 1960 in the unconstrained configuration is approximately between 3% and 8% of the total length of the device in the unconstrained configuration.

The braided section between second intermediate edge 1960 and the proximal inflection section 1970 may define a second intermediate plane (or a distal cone when considering the device as a three dimensional structure), the sending intermediate plane or cone having a first tangency near the edge 1960 and a second tangency near the inflection section 1970. In some embodiments, the proximal tier 1980 comprises a braided structure, the braided structure defining an upper or distal plane and a lower or proximal plane wherein the distal plane may be approximately parallel to the proximal plane, wherein the upper plane may be approximately perpendicular to the central axis Z of the device. In some embodiments, the upper plane defined by the proximal tier 1980 may intersect with the second intermediate plane near the proximal inflection section 1970, thereby defining an angle between the two planes. In some embodiments, the angle between the upper proximal plane and the second intermediate plane may be between 2 degrees to 80 degrees. In some embodiments, the angle between the upper proximal plane and the second intermediate plane may be between 3 degrees to 45 degrees. In some embodiments, the angle between the upper proximal plane and the second intermediate plane may be between 10 degrees to 45 degrees. In some embodiments, the angle between the upper proximal plane and the second intermediate plane may be between 20 degrees to 60 degrees.

In some embodiments, the disk-shaped proximal tier 1980 comprises a braid comprising pores having a first pore density and the intermediate section 1955 comprises pores having a second pore density. In some embodiments, the first pore density of the proximal tier 1980 is greater than the pore density of the second pore density of the intermediate tier 1955. In some embodiments, at least one pore in the intermediate tier 1955 has a longitudinal axis and a circumferential axis, wherein the length of the pore in the longitudinal axis is greater than the length of the of the same pore in the circumferential axis. In some embodiments, at least one pore in the proximal tier 1980 has a longitudinal axis and a circumferential axis, wherein the length of the pore in the longitudinal axis is less than the length of the of the same pore in the circumferential axis.

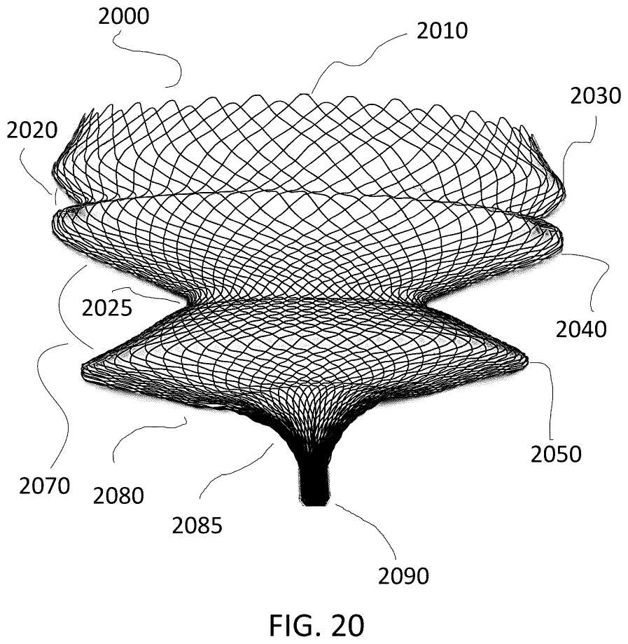

FIG. 20 shows an embodiment of an implantable device 2000, the device comprising a single layer of braided material with an open distal end 2010 comprising loops or apices, and a distal opening defining a first diameter. A second section, adjacent to the distal opening, comprises a bend or curve 2030 wherein the curve 2030 defines a second diameter. In some embodiments, the second diameter may be larger than the first diameter. In some embodiments, the second diameter may be 5-15% larger than the first diameter. In some embodiments, the second diameter may be 10-30% larger than the first diameter. A third section, adjacent to the second section, comprises a bend or curve 2040 and comprises a third diameter defined by the bend 2040. In some embodiments, the third diameter is approximately equal to the second diameter. In some embodiments, the third diameter is approximately 0.1-1.0 mm smaller than the second diameter. In some embodiments, the third diameter is approximately 5-30% smaller than the second diameter. A first waist section between the bends 2030 and 2040 defines a fourth diameter and an angle 2020. The height of the first waist section may be defined by a longitudinal length L (see FIG. 2) of the pore(s) that comprise the first waist section. In some embodiments, the height of the first waist section is less than the length L of a single pore. In some embodiments, the height of the first waist section is approximately equal to the length L of a single pore. In some embodiments, the height of the first waist section is approximately equal to at least the length L of two pores. In some embodiments, the height of the first waist section is approximately 0.5×L to 4×L. In some embodiments, the angle 2020 is approximately 5 degrees to 45 degrees. In some embodiments, the angle 2020 is approximately 5 degrees to 60 degrees. In some embodiments, the angle 2020 is approximately 5 degrees to 15 degrees. In some embodiments, the fourth diameter is approximately 0.5 mm to 2 mm smaller than the third diameter. In some embodiments, the fourth diameter is approximately 5-15% smaller than the third diameter. In some embodiments, the fourth diameter is approximately 10-40% smaller than the third diameter. A fourth section, adjacent to the third section, is between the bend 2040 and a bend near 2050, wherein the bend 2050 defines a fifth diameter. In some embodiments, the fifth diameter may be approximately equal to the first diameter. In some embodiments, the fifth diameter may be approximately equal to the second diameter. In some embodiments, the fifth diameter may be approximately equal to the third diameter. In some embodiments, the fifth diameter may be approximately 0.5 mm to 5 mm smaller than the second diameter. In some embodiments, the fifth diameter may be approximately 0.5 mm to 2 mm smaller than the second diameter. In some embodiments, the fifth diameter may be approximately 5-40% smaller than the second diameter. The fourth section may have a shape approximating two trapezoidal or frustoconical sections lying on top of each other. The fourth section may comprise a second waist 2025 having a sixth diameter. In some embodiments, the sixth diameter is approximately 10-60% smaller than the fifth diameter. In some embodiments, the sixth diameter is approximately 30-50% smaller than the fifth diameter. In some embodiments, the sixth diameter is approximately 0.5 mm to 5 mm smaller than the fifth diameter. In some embodiments, the sixth diameter is approximately 2 mm to 4 mm smaller than the fifth diameter. In some embodiments, the second waist section comprises an angle 2070 between the upper and lower trapezoidal or frustoconical sections of the fourth section. In some embodiments, the angle 2070 is approximately 10-60 degrees. In some embodiments, the angle 2070 is approximately 15-45 degrees. The height of the second waist section may be defined by a longitudinal length L (see FIG. 2) of the pore(s) that comprise the second waist section. In some embodiments, the height of the second waist section is less than the length L of a single pore. In some embodiments, the height of the second waist section is approximately equal to the length L of a single pore. In some embodiments, the height of the second waist section is approximately equal to at least the length L of two pores. In some embodiments, the height of the second waist section is approximately 0.5×L to 4×L. The device 2000 further comprises a proximal section 2080 between the bend 2050 and a cylindrical gathering section 2090. The proximal section 2080 may have an approximately flat plane or have a slightly convex shape, or a slightly concave shape. The proximal section 2080 may form a sloping, approximately straight section transitioning to an arc or arced section 2085. In some embodiments, the arc or arced section 2085 may form the approximate shape of a funnel. In some embodiments, the proximal gathering section 2090 may be in the form of a cylindrical metallic or plastic tube in which elongate filaments forming the braided structure may be held in place by, for example, soldering, welding, laser welding, crimping, or adhesive bonding. In some embodiments, the overall length of the device 2000 measured from a plane approximately perpendicular to the apices of the loops 2010 to the proximal end of the gathering section 2090 is approximately 30% to 60% of the second diameter. In some embodiments, the proximal gathering section 2090 may comprise an opening or aperture configured to hold a detachment tether or wire that connects the implant to a delivery system.

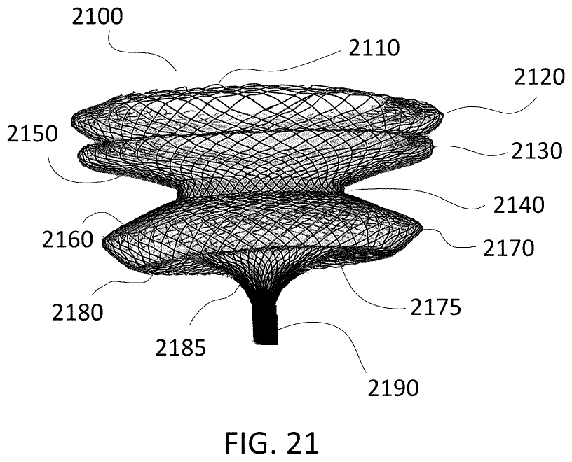

FIG. 21 shows an embodiment of an implantable device 2100, the device comprising a single layer of braided material with an open distal end 2110 comprising loops or apices folded in the direction of a longitudinal central axis and a distal opening defining a first diameter. A second section, adjacent to the distal opening, comprises a bend or curve 2120 wherein the curve 2120 defines a second diameter. In some embodiments, the second diameter may be larger than the first diameter. In some embodiments, the second diameter may be 30-80% larger than the first diameter. In some embodiments, the second diameter may be 10-50% larger than the first diameter. A third section, adjacent to the second section, comprises a bend or curve 2130 and comprises a third diameter defined by the bend 2130. In some embodiments, the third diameter is approximately equal to the second diameter. In some embodiments, the third diameter is approximately 0.1-3 mm smaller than the second diameter. In some embodiments, the third diameter is approximately 1-30% smaller than the second diameter. A first waist section between the bends 2120 and 2130 defines a fourth diameter. The height of the first waist section may be defined by a longitudinal length L (see FIG. 2) of the pore(s) that comprise the first waist section. In some embodiments, the height of the first waist section is less than the length L of a single pore. In some embodiments, the height of the first waist section is approximately equal to the length L of a single pore. In some embodiments, the height of the first waist section is approximately equal to at least the length L of two pores. In some embodiments, the height of the first waist section is approximately 0.1×L to 2×L. In some embodiments, the fourth diameter is approximately 0.5 mm to 3 mm smaller than the third diameter. In some embodiments, the fourth diameter is approximately 1-15% smaller than the third diameter. In some embodiments, the fourth diameter is approximately 5-40% smaller than the third diameter. A fourth section, adjacent to the third section, is between the bend near 2130 and a bend near 2170, wherein the bend 2170 defines a fifth diameter. In some embodiments, the fifth diameter may be approximately equal to the second diameter. In some embodiments, the fifth diameter may be approximately equal to the third diameter. In some embodiments, the fifth diameter may be approximately 0.5 mm to 5 mm smaller than the second diameter. In some embodiments, the fifth diameter may be approximately 0.5 mm to 2 mm smaller than the second diameter. In some embodiments, the fifth diameter may be approximately 5-30% smaller than the second diameter. In some embodiments, the fourth section comprises a first proximal-facing surface 2150, a second distal-facing surface 2160 and a second waist section 2140 therebetween. The fourth section may have a shape approximating two trapezoidal or frustoconical sections lying on top of each other. In some embodiments, the height of the second waist section 2140 is less than the length L of a single pore. In some embodiments, the height of the second waist section is approximately equal to the length L of a single pore. In some embodiments, the height of the second waist section is approximately equal to at least the length L of two pores. In some embodiments, the height of the second waist section is approximately 0.5×L to 4×L. The device 2100 further comprises a proximal plane 2180 comprising a bevel-shaped lip approximately between the bend 2170 and the proximal plane 2180 wherein the bevel-shaped lip comprises pores having a height L and wherein the bevel-shaped lip has a height of 0.5×L to 4×L. The proximal plane 2180 may comprise a bend creating an upward sloping (concave) surface to a transition area 2175. At or near the transition area 2175, the braid may change direction to a downward sloping surface 2185. In some embodiments, a 3-D rotation of the surface 2185 may form the approximate shape of a funnel. In some embodiments, the proximal gathering section 2190 may be in the form of a cylindrical metallic or plastic tube in which elongate filaments forming the braided structure may be held in place by, for example, soldering, welding, laser welding, crimping, or adhesive bonding. In some embodiments, the proximal gathering section 2190 may comprise an opening or aperture configured to hold a detachment tether or wire that connects the implant to a delivery system.

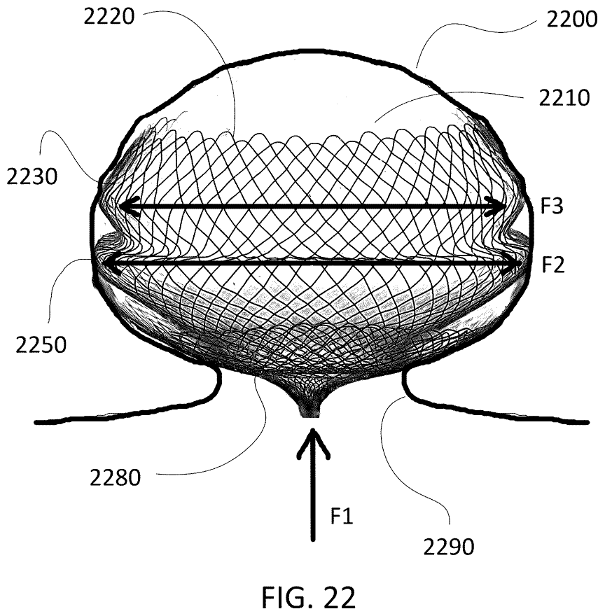

FIG. 22 shows a non-limiting illustrative example of a device 2210 of the previous embodiments deployed in an aneurysm 2200 having a wall 2250 and a neck 2290. The device 2210 comprises a looped distal end 2220 and a proximal section 2280. The axial force vector (F1) of the blood flow acting on the proximal section 2280 shown along with the radial force vectors (F2 and F3, shown for illustrative purposes, the force vector could be combined into one vector or shown as multiple vectors) exerted by the device 2210 on the aneurysm wall 2250. A unique and expected result occurs due to the design of the device 2210. Specifically, as F1 increases, a portion of the axial force of the blood flow is converted to increased radial force F2 and/or F3. Thus, as the axial force F1 of the blood flow increases, the device 2210 becomes more tightly wedged within the aneurysm 2200 by increasing radial force F2 and/or F3. This feature makes the device 2210 more stable within the aneurysm 2200 and less likely to move or compact over time.

Some of the embodiments described above comprise a method for treating an aneurysm or blood vessel, the method comprising:
a) providing an implantable device, the device having an unconstrained configuration, a constrained configuration within a delivery catheter having a distal end and a proximal end, and a deployed configuration within the aneurysm or blood vessel, wherein:
   i) in the unconstrained configuration, the device comprises a single layer of braided or resilient mesh material, an open distal end, and a closed proximal end; and
   ii) in the unconstrained configuration, the device comprises a saucer-shaped or disc-shaped section, a cylinder-shaped section, and a conical-shaped or frustoconical-shaped section; and
   iii) in the constrained configuration, the device comprises a resilient mesh or braid configured to pass from the proximal end of the delivery catheter to the distal end of the delivery catheter;
b) maneuvering the distal end of the delivery catheter to the aneurysm or blood vessel; and
c) deploying the device out of the distal end of the delivery catheter, wherein the device is configured to fold upon itself during delivery.
   In some embodiments, the implantable mesh device may be configured so that a distal disc-shaped or saucer-shaped section folds over an intermediate cylindrical section such that at least a portion of the distal end of the device is oriented toward the dome of an aneurysm. In some embodiments, the implantable mesh device may be configured so that, during deployment, a proximal portion of the device compresses or folds near the neck of the aneurysm or within a blood vessel to form multiple layers. In some embodiments, the device is configured so that, depending on the geometry of the treatment site, a distal section of the device may fold over an intermediate section, a proximal section may fold or compress to form multiple layers, or both distal and proximal sections may fold or compress to form multiple layers.

### Recitals:

1. a Device for Treatment of an Aneurysm Within a Patient's Vasculature, comprising:
   a self-expanding resilient permeable shell comprising a substantially closed proximal end and a substantially open distal section; the shell comprising a plurality of elongate resilient filaments having a braided structure,
   wherein the permeable shell is a single layer of braided elongate resilient filaments,
   wherein the permeable shell has a radially constrained state configured for delivery within a microcatheter,
   wherein the permeable shell has an expanded state,
   wherein the distal section in the expanded state has a plurality of pores having a first density,
   wherein the permeable shell comprises an intermediate section proximal to the distal section and distal to the proximal end, the intermediate section in the expanded state having a plurality of pores having a second density, and
   wherein the first density of the plurality of pores in the distal section is greater than the second density of the plurality of pores in the intermediate section.
2. The device of recital 1 wherein the permeable shell in its expanded state has a region of maximum diameter within the distal section.
3. The device of recital 1 wherein the permeable shell in its expanded state has a substantially open distal end, the distal end comprising a plurality of loops of elongate resilient filaments.
4. The device of recital 1 wherein a pore in the distal section has a circumferential length and a longitudinal length and a pore in the intermediate section has a circumferential length and a longitudinal length, wherein the circumferential length of the pore in the distal section is greater than the circumferential length of the pore in the intermediate section and the longitudinal length of the pore in the intermediate section is longer than the longitudinal length of the pore in the distal section.
5. The device of recital 1 wherein the device is formed from 40-400 nickel titanium alloy wires ranging from 0.0004 to 0.003 inches diameter and having a radiopaque core material comprising 10%-40% of the wires'cross sectional area.
6. The device of recital 1 wherein the permeable shell in the expanded state comprises a proximal section having a third pore density, wherein the proximal section comprises a flange, and wherein the third pore density is greater than the second pore density.
7. A method for treating an aneurysm having an equatorial region and a neck region, the method comprising: deploying within the aneurysm, a device comprising:
   (i) a resilient mesh structure formed from one or more filaments, the structure having a delivery shape and a deployed shape capable of conforming to the aneurysm walls;
   (ii) a single layer of resilient braided mesh having a substantially open distal end defining a first circumference and a substantially closed proximal end having a gathering section;
   (iii) a delivery system detachably coupled to the gathering section;
   (iv) a flange section defining a second circumference wherein the second circumference is configured to substantially conform the neck region of the aneurysm wherein the flange comprises an upper layer and a lower layer and a gap between the upper layer and the lower layer; and the method further comprising the steps of:
      (a) deploying the distal end of the device near the equatorial region of the aneurysm;
      (b) deploying the flange of the device near the neck region of the aneurysm;
      (c) placing tension on the delivery system to decrease the gap between the upper layer and lower layer of the flange; and
      (d) detaching the gathering section from the delivery system.
8. The method of recital 7 wherein the device is formed from 32-400 nickel titanium alloy wires ranging from 0.0004 to 0.003 inches diameter and having a radiopaque core material comprising 10%-40% of the wires'cross sectional area.
9. The method of recital 7 wherein the flange is proximally biased when the device is in an expanded state and configured to flip into a distally biased orientation.
10. The method of recital 7 wherein the device in the delivery shape has a first length and wherein the device in the deployed state has a second length wherein the first length is longer than the second length.
11. The method of recital 7 wherein the deployed shape comprises at least two overlapping layers of resilient mesh structure near the aneurysm neck region comprising a portion of the upper layer of the flange and a portion of the lower layer of the flange.
12. The method of recital 7 wherein the device comprises a resilient mesh intermediate section and wherein the deployed shape comprises at least three overlapping layers of resilient mesh structure near the aneurysm neck region, the overlapping layers comprising a portion of the intermediate section, a portion of the upper layer of the flange, and a portion of the lower layer of the flange.
13. The method of recital 7 wherein the flange comprises a rim, overhang, projection, extension, lip, or protuberance of resilient braided mesh material.
14. A single-layer braided device for treating an aneurysm, the aneurysm having an equatorial region, wherein the device has a deployed configuration, the device comprising:
   an open distal end comprising castellated loops configured for deployment near the equatorial region of the aneurysm;
   a first section proximal to the distal end;
   a second section proximal to the first section;
   a third section proximal to the second section;
   a closed proximal end; and
   wherein the first section comprises braided filaments formed into a disc-shaped structure, the second section comprises braided filaments formed into a cylinder-shaped structure, and the third section comprises braided filaments configured to form multiple folded sections in the deployed configuration.
15. The device of recital 14 wherein in the deployed configuration the first section is configured to flip at least partially over the second section and wherein at least one of the distal castellated loops comprising the distal end is distally oriented after the first section at least partially flips over the second section.
16. The device of recital 14 wherein the device comprises a central axis and an unconstrained configuration wherein in the unconstrained configuration at least one of the loops comprising the distal end are oriented approximately perpendicular to the central axis.
17. The device of recital 14 wherein the braided filaments comprising the third section form non-parallel overlaps in the deployed configuration.
18. The device of recital 14 wherein the braided filaments forming the second section comprise diamond-shaped pores, at least one pore in the second section having a first circumferential length and a first longitudinal length, and wherein the braided filaments forming the third section comprise diamond-shaped pores, at least one pore in the third section having a second circumferential length and a second longitudinal length wherein the first longitudinal length is longer than the first circumferential length and wherein the second circumferential length is longer than the second longitudinal length.
19. A medical device having a resilient mesh braided structure, the device comprising:
   an open distal end comprising a series of loops, a closed proximal end;
   a disc-shaped distal tier comprising braided filaments proximal to the distal end;
   a cylinder-shaped intermediate tier comprising of braided filaments proximal to the distal tier;
   a first inflection section between the distal tier and the intermediate tier wherein the first inflection section defines an angle between the distal tier and the intermediate tier;
   a disc-shaped proximal tier comprising of braided filaments proximal to the intermediate tier and distal to the proximal end; and
   wherein the angle between the distal tier and the intermediate tier is between 15 degrees and 60 degrees.
20. The device of recital 19 wherein the device comprises a central axis and an unconstrained configuration wherein in the unconstrained configuration at least 50% of the loops comprising the distal end are oriented approximately perpendicular to the central axis.
21. The device of recital 19 wherein the braided filaments forming the intermediate tier comprise diamond-shaped pores, at least one pore in the intermediate tier having a first circumferential length and a first longitudinal length, and wherein the braided filaments forming the proximal tier comprise diamond-shaped pores, at least one pore in the proximal tier having a second circumferential length and a second longitudinal length wherein the first longitudinal length is longer than the first circumferential length and wherein the second circumferential length is longer than the second longitudinal length.
22. The device of recital 19 wherein the device comprises a central axis and an unconstrained configuration wherein in the unconstrained configuration at
least one of the loops comprising the distal end are oriented approximately perpendicular to the central axis.
23. The device of recital 19 wherein the device comprises a central axis and an unconstrained configuration wherein in the unconstrained configuration at least 50% of the loops comprising the distal end are oriented approximately perpendicular to the central axis.

A number of embodiments of the invention have been described. Without departing from the scope and spirit of the present invention, reasonable features, modifications, advantages, and design variable of the claimed apparatus will become readily apparent to those skilled in the art by following the guidelines set forth in the preceding detailed description and embodiments. Accordingly, other embodiments are within the scope of the following claims.

1. A device for treatment of an aneurysm within a patient's vasculature, comprising:
a self-expanding resilient permeable shell comprising a substantially closed proximal end having a longitudinal axis and a substantially open distal section; the shell comprising a plurality of elongate resilient filaments having a single-layer braided structure; and
a first intermediate section between the distal section and the proximal end, said first intermediate section having a first diameter,
a second intermediate section between the first intermediate section and the proximal end, said second intermediate section having a second diameter,
a proximal section between the second intermediate section and the proximal end, said proximal section having a third diameter,
wherein the second diameter is at least 5% smaller than the first diameter, and wherein the third diameter is at least 10% smaller than the first diameter.

2. The device of claim 1 wherein the distal section comprises a series of loops having apices, wherein the proximal end has a longitudinal axis, and wherein at least one of the apices is approximately parallel to the longitudinal axis.

3. The device of claim 1 comprising a waist section between the second intermediate section and the proximal section, the waist section having a height and the waist section formed from braid having a longitudinal pore length L, wherein the height is less three times L.

4. The device of claim 1 wherein the distal section comprises a series of loops having apices wherein the length of the device from at least one of the apices to the proximal end is between 30% and 60% of the first diameter.

5. The device of claim 1 wherein the proximal end comprises an approximately cylindrical tube wherein the elongate resilient filaments are gathered within the cylindrical tube and wherein the cylindrical tube comprises an aperture configured to hold a detachment tether configured to connect the device to a delivery system.

6. The device of claim 1 wherein the second diameter is 5% to 20% smaller than the first diameter, and wherein the third diameter is 10% to 25% smaller than the first diameter.

7. A method for treating an aneurysm having an equatorial region and a neck region, the method comprising: deploying within the aneurysm, a device comprising:
(i) a resilient mesh structure formed from one or more filaments, the structure having a delivery shape and a deployed shape capable of conforming to the aneurysm walls;
(ii) a single layer of resilient braided mesh having a substantially open distal end defining a first circumference and a substantially closed proximal end having a gathering section;
(iii) a delivery system detachably coupled to the gathering section;
(iv) a flange section defining a second circumference wherein the second circumference is configured to substantially conform the neck region of the aneurysm wherein the flange comprises an upper layer and a lower layer and a gap between the upper layer and the lower layer; and the method further comprising the steps of:
(a) deploying the distal end of the device near the equatorial region of the aneurysm;
(b) deploying the flange of the device near the neck region of the aneurysm;
(d) detaching the gathering section from the delivery system.

8. The method of claim 7 wherein the device is formed from 32-400 nickel titanium alloy wires ranging from 0.0004 to 0.003 inches diameter and having a radiopaque core material comprising 10%-40% of the wires'cross sectional area.

9. The method of claim 7 wherein the flange comprises a braided surface, said surface incorporating at least one upward sloping portion and at least one downward sloping portion.

10. The method of claim 7 wherein the device in the delivery shape has a first length and wherein the device in the deployed state has a second length wherein the first length is longer than the second length.

11. The method of claim 7 wherein the deployed shape comprises at least two overlapping layers of resilient mesh structure near the aneurysm neck region comprising a portion of the upper layer of the flange and a portion of the lower layer of the flange.

12. The method of claim 7 wherein the device comprises a resilient mesh intermediate section and wherein the deployed shape comprises at least two overlapping layers of resilient mesh structure near the aneurysm neck region, the overlapping layers comprising a portion of the upper layer of the flange and a portion of the lower layer of the flange.

13. The method of claim 7 wherein the flange comprises a rim, overhang, projection, extension, lip, bevel-shaped lip, or protuberance of resilient braided mesh material.

14. A single-layer braided device for treating an aneurysm, the aneurysm having a wall, wherein the device has a deployed configuration, the device comprising:
an open distal end comprising castellated loops;
a first section proximal to the distal end;
a second section proximal to the first section;
a third section proximal to the second section;
a closed proximal end; and
wherein the first section comprises braided filaments formed into a disc-shaped structure, the second section comprises braided filaments formed into a cylinder-shaped structure, and the third section comprises braided filaments configured to form multiple folded sections in the deployed configuration.

15. The device of claim 14 wherein in the deployed configuration the first section is configured to flip at least partially over the second section and wherein at least one of the distal castellated loops comprising the distal end is distally oriented after the first section at least partially flips over the second section.

16. The device of claim 14 wherein the device comprises a central axis and an unconstrained configuration wherein in the unconstrained configuration at least one of the loops comprising the distal end are oriented approximately perpendicular to the central axis.

17. The device of claim 14 wherein the device is subjected to blood flow in an axis perpendicular to the proximal end, said blood flow generating an axial force, wherein a portion of said axial force is converted to a radial force perpendicular to the wall of the aneurysm through one or more of said sections and wherein an increase to the axial force causes an increase to the radial force.

18. The device of claim 14 wherein the braided filaments forming the second section comprise diamond-shaped pores, at least one pore in the second section having a first circumferential length and a first longitudinal length, and wherein the braided filaments forming the third section comprise diamond-shaped pores, at least one pore in the third section having a second circumferential length and a second longitudinal length wherein the first longitudinal length is longer than the first circumferential length and wherein the second circumferential length is longer than the second longitudinal length.
